(19) United States
(12) Patent Application Publication
(10) Pub. No.: US 2025/0325322 A1
(43) Pub. Date: Oct. 23, 2025

(54) PULSED-FIELD ABLATION AND MAPPING SYSTEM

(71) Applicant: CRC EP, INC., Lake Oswego, OR (US)

(72) Inventors: Dorin PANESCU, San Jose, CA (US); Tho NGUYEN, Huntington Beach, CA (US); Henning EBERT, Berlin (DE); Steffen HOLZINGER, Berlin (DE); Sven BODE, Berlin (DE)

(21) Appl. No.: 19/260,045

(22) Filed: Jul. 3, 2025

Related U.S. Application Data
(63) Continuation of application No. 19/028,908, filed on Jan. 17, 2025.
Application No. 19/028,908 is a continuation of application No. 18/604,179, filed on Mar. 13, 2024, now Pat. No. 12,201,344.
Application No. 18/604,179 is a continuation of application No. PCT/US2023/012201, filed on Feb. 2, 2023.
(60) Provisional application No. 63/306,162, filed on Feb. 3, 2022.

(51) Int. Cl. A61B 18/12; A61B 18/00; A61B 18/14; A61B 34/00

(52) U.S. Cl. CPC A61B 18/1206; A61B 18/1492; A61B 34/25; A61B 2018/00577; A61B 2018/00613; A61B 2018/00767; A61B 2018/00827; A61B 2018/00875; A61B 2018/00892; A61B 2018/126; A61B 2018/1286

(57) ABSTRACT

The present invention relates to one or more mapping electrodes that reside on a PFA catheter connected to a generator (G) capable of transferring the mapping signals to a mapping system and of providing PFA energy to the catheter electrodes for cardiac tissue treatment purposes. Further aspects relate to a catheter, a system, a method.

### RELATED APPLICATION

The present application is a continuation of U.S. application Ser. No. 19/028,908, filed 17 Jan. 2025, which is a continuation of U.S. application Ser. No. 18/604,179, filed 13 Mar. 2024, which is a continuation of International Patent Application No. PCT/US2023/012201 filed 2 Feb. 2023, which claims priority to U.S. provisional patent application No. 63/306,162 filed 3 Feb. 2022, each of which is incorporated herein by reference in its entirety.

### BACKGROUND

### Field of the Invention

The present invention relates to systems and methods to map intracardiac activity and to generate, to deliver and to perform pulsed-field ablation with, charge-balanced waveforms.

### Related Art

In the medical field, various methods and medical devices for treating a tissue based on an electrical energy and/or power are known. For example, the electrical energy/power may be used for ablating a tissue. A tissue ablation may be performed for treating and/or preventing various diseases. For example, it is known to ablate cardiac tissue for treating cardiovascular diseases (e.g., cardiac arrythmias, such as atrial fibrillation, ventricular tachycardia, etc.). The medical device in this case may, for example, be an ablation catheter. However, also other types of tissues may be treated based on an electrical energy for medical purposes with other types of medical devices.

To enable a reliable treatment, the application or source of the electrical energy/power for the tissue treatment usually needs to be controlled in a defined way to ensure a desired medical outcome for the patient. In addition to reliable treatment, minimal, or no, damage to adjacent tissue structure is also required. For example, it is known that radiofrequency ablation may produce damage to the esophagus. In some case, an atrial-esophageal fistula develops. Such condition may be life threatening. Additionally, mapping can more precisely target the tissue region that should be ablated.

Therefore, energy modalities that spare collateral tissues are desired. For example, a pulsed-field ablation (PFA) treatment is known to spare the esophagus, phrenic nerves, coronary structures, etc. However, if not designed optimally, PFA waveforms may cause significant skeletal muscle stimulation, which can be painful, or microbubbling, which may result in embolic events.

Therefore, there is a need to find ways to combine intracardiac mapping with PFA energy generation for treatment of cardiac conditions.

### SUMMARY

The aspects described herein address the above need at least in part.

In some aspects, the techniques described herein relate to an ablation system for treatment of patient tissue, including: an ablation catheter including an ablation portion having a plurality of electrodes at a distal end, wherein the ablation portion includes at least two loop sections forming a three dimensional spiral, a generator adapted to connect to a proximal end of the ablation catheter to electrically couple to the plurality of electrodes, the generator configured to energize one or more of the plurality of electrodes with high-voltage charge-balanced pulsed electric fields and receive electrical signals from one or more of the plurality of electrodes; and the generator including an interface configured to connect to an external device and further configured to protect the external device from a high voltage pulse.

In some aspects, the techniques described herein relate to an ablation system, wherein the external device includes a mapping system.

In some aspects, the techniques described herein relate to an ablation system, wherein the external device includes a recording system.

In some aspects, the techniques described herein relate to an ablation system, wherein the external device includes an electro-anatomical mapping system.

In some aspects, the techniques described herein relate to an ablation system, wherein the electro-anatomical mapping system includes a recording system.

In some aspects, the techniques described herein relate to an ablation system, wherein the interface is configured to protect the external system from a high voltage pulse.

In some aspects, the techniques described herein relate to an ablation system, wherein the interface includes one or more voltage suppressors configured to protect the external system from a high voltage pulse.

In some aspects, the techniques described herein relate to an ablation system, wherein the interface includes one or more switches configured to protect the external system from a high voltage pulse.

In some aspects, the techniques described herein relate to an ablation system, wherein the interface includes one or more relays configured to protect the external system from a high voltage pulse.

In some aspects, the techniques described herein relate to an ablation system, wherein the generator further includes an electronic control unit adapted to switch between an ablation mode and a mapping mode for each of the plurality of electrodes.

In some aspects, the techniques described herein relate to an ablation system, wherein electrodes used for ablation in the ablation mode are used for mapping in the mapping mode.

In some aspects, the techniques described herein relate to an ablation system, wherein a distance between a first electrode on a first loop section having a first polarity and a second electrode on a second loop section having a second polarity opposite the first polarity and closest to the first electrode results in an arcing risk index less than 0.25 in a compressed position where the ablation portion is flattened by an external force and an arcing risk index less than 0.25 in an uncompressed position where the ablation portion is not restricted by any external force.

In some aspects, the techniques described herein relate to an ablation system, wherein a distance between a first electrode on a first loop section having a first polarity and a second electrode on a second loop section having a second polarity opposite the first polarity and closest to the first electrode results in an arcing risk index less than 0.15 in a compressed position where the ablation portion is flattened by an external force and an arcing risk index less than 0.15 in an uncompressed position where the ablation portion is not restricted by any external force.

In some aspects, the techniques described herein relate to an ablation system for treatment of patient tissue, including: an ablation catheter including an ablation portion having a plurality of electrodes at a distal end, wherein the ablation portion includes at least two loop sections forming a three dimensional spiral, a generator adapted to connect to a proximal end of the ablation catheter to electrically couple to the plurality of electrodes, the generator configured to energize one or more of the plurality of electrodes with high-voltage charge-balanced pulsed electric fields and receive electrical signals from one or more of the plurality of electrodes; and an external device, wherein the generator includes an interface configured to connect to the external device further configured to protect the external device from a high voltage pulse.

In some aspects, the techniques described herein relate to an ablation system, wherein the external device includes a mapping system.

In some aspects, the techniques described herein relate to an ablation system, wherein the external device includes a recording system.

In some aspects, the techniques described herein relate to an ablation system, wherein the external device includes an electro-anatomical mapping system.

In some aspects, the techniques described herein relate to an ablation system, wherein the electro-anatomical mapping system includes a recording system.

In some aspects, the techniques described herein relate to an ablation system, wherein the interface is configured to protect the external system from a high voltage pulse.

In some aspects, the techniques described herein relate to an ablation system, wherein the interface includes one or more voltage suppressors configured to protect the external system from a high voltage pulse.

In some aspects, the techniques described herein relate to an ablation system, wherein the interface includes one or more switches configured to protect the external system from a high voltage pulse.

In some aspects, the techniques described herein relate to an ablation system, wherein the interface includes one or more relays configured to protect the external system from a high voltage pulse.

In some aspects, the techniques described herein relate to an ablation system, wherein the generator further includes an electronic control unit adapted to switch between an ablation mode and a mapping mode for each of the plurality of electrodes.

In some aspects, the techniques described herein relate to an ablation system, wherein electrodes used for ablation in the ablation mode are used for mapping in the mapping mode.

In some aspects, the techniques described herein relate to an ablation system, wherein a distance between a first electrode on a first loop section having a first polarity and a second electrode on a second loop section having a second polarity opposite the first polarity and closest to the first electrode results in an arcing risk index less than 0.25 in a compressed position where the ablation portion is flattened by an external force and an arcing risk index less than 0.25 in an uncompressed position where the ablation portion is not restricted by any external force.

In some aspects, the techniques described herein relate to an ablation system, wherein a distance between a first electrode on a first loop section having a first polarity and a second electrode on a second loop section having a second polarity opposite the first polarity and closest to the first electrode results in an arcing risk index less than 0.25 in a compressed position where the ablation portion is flattened by an external force and an arcing risk index less than 0.25 in an uncompressed position where the ablation portion is not restricted by any external force.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

In the following, the Figures of the present disclosure are listed:

FIG. 1 depicts a distal end of a first embodiment of an ablation catheter in a perspective side view;

FIG. 2 illustrates a delivery path for an ablation catheter leading to a pulmonary vein ostium of a human heart;

FIG. 3 and FIG. 3A show part of the electric control of the electrode leads for the embodiment of the ablation catheter of FIG. 1;

FIG. 4 depicts the distal end of the ablation catheter of FIG. 1 with electrode numbering in a top view;

FIG. 5 and FIG. 6 show matrices containing AR indexes for each electrode pair and the impedance values of the ablation catheter of FIG. 1 for a saline position of the ablation portion;

FIG. 7 shows the ablation portion of the ablation catheter of FIG. 1 pressed to chicken heart tissue in a top view;

FIG. 8 and FIG. 9 show matrices containing AR indexes for each electrode pair and the CU value of the ablation catheter of FIG. 1 in the position shown in FIG. 7;

FIG. 10 shows another position of the ablation portion of the ablation catheter of FIG. 1 pressed to chicken heart tissue in a top view;

FIG. 11 and FIG. 12 show matrices containing AR indexes for each electrode pair and the CU value of the ablation catheter of FIG. 1 in the position shown in FIG. 10;

FIG. 13 depicts a schematic example of an applicable PFA waveform;

FIG. 14 shows another position of the ablation portion of the ablation catheter of FIG. 1 pressed to a chicken heart tissue in a top view;

FIG. 15 shows a matrix containing impedance values and an AR index (flagged) for the electrode pairs 1,2; 2,3; 2,9; 8,9; 9,10, respectively of the ablation catheter of FIG. 1 in the position shown in FIG. 14;

FIG. 16 shows another position of the ablation portion of the ablation catheter of FIG. 1 pressed to a chicken heart tissue in a top view;

FIG. 17 shows a matrix containing impedance values and an AR index (flagged) for the electrode pairs 1,2; 2,3; 2,9; 8,9; 9,10, respectively of the ablation catheter of FIG. 1 in the position shown in FIG. 16;

FIG. 18 shows another position of the ablation portion of the ablation catheter of FIG. 1 pressed to a chicken heart tissue in a top view;

FIG. 19 shows a matrix containing impedance values and an AR index (flagged) for the electrode pairs 1,2; 2,3; 2,9; 8,9; 9,10, respectively of the ablation catheter of FIG. 1 in the position shown in FIG. 18;

FIG. 20 shows another position of the ablation portion of the ablation catheter of FIG. 1 pressed to a chicken heart tissue in a top view;

FIG. 20A shows another position of the ablation portion of the ablation catheter of FIG. 1 pressed to a chicken heart tissue in a top view;

FIG. 21 shows a matrix containing impedance values for the adjoining electrode pairs derived from a bipolar measurement and a CU value of the ablation catheter of FIG. 1 in the position shown in FIG. 20 calculated from these impedance values;

FIG. 21A shows a matrix containing impedance values for the adjoining electrode pairs derived from a bipolar measurement and a CU value of the ablation catheter of FIG. 1 in the position shown in FIG. 20A calculated from these impedance values;

FIG. 22 shows a table containing impedance values derived from a quasi-unipolar measurement at 500 kHz and a CU value calculated from these impedance values of the ablation catheter of FIG. 1 in the position shown in FIG. 20;

FIG. 22A shows a table containing impedance values derived from a quasi-unipolar measurement at 500 kHz and a CU value calculated from these impedance values of the ablation catheter of FIG. 1 in the position shown in FIG. 20A;

FIG. 23, FIG. 24, and FIG. 25 visualize three different pulse shapes for current measurements at each individual electrode;

FIG. 26, FIG. 27, FIG. 28, and FIG. 29 show four different positions of the ablation catheter of FIG. 1, partly with respect to a chicken heart tissue in saline;

FIG. 30 shows a bar diagram containing impedance values determined for the four positions of FIGS. 26 to 29 with respect to each electrode of the ablation portion of the ablation catheter of FIG. 1;

FIG. 31 shows a position of the ablation catheter of FIG. 1 within a heart of an 80 kg pig;

FIG. 32 shows a bar diagram containing impedance values determined for the position of the catheter depicted in FIG. 31 with respect to each electrode of the ablation portion of the catheter.

FIG. 33 visualizes a flowchart for the use of a PFA catheter including PFA precheck determining AR indexes and CU value in order to treat paroxysmal atrial fibrillation; and

FIG. 34 and FIG. 35 show examples of visualization of impedance values for electrodes of an ablation section of an ablation catheter similar to the one of FIG. 1.

FIG. 36: Schematic representation of an exemplary embodiment of a generator according to the present invention.

FIG. 37: Schematic representation of an output relay board of the exemplary embodiment of the generator.

FIG. 38: Schematic representation of various components of the exemplary embodiment of the generator.

FIG. 39A and FIG. 39B: Representation of a first example of a charge-balanced voltage and current pulse implemented by a generator according to the invention.

FIG. 40A and FIG. 40B: Representation of a second example of a charge-balanced voltage and current pulse implemented by a generator according to the invention.

FIG. 41A and FIG. 41B: Representation of a third example of a charge-balanced voltage and current pulse implemented by a generator according to the invention.

FIG. 42: Representation of a fourth example of a charge-balanced voltage and current pulse implemented by a generator according to the invention.

FIG. 43: Representation of an exemplary user interface of a generator according to the invention.

### DETAILED DESCRIPTION

Subsequently, presently preferred embodiments will be outlined, primarily with reference to the above Figures. It is noted that further embodiments are certainly possible, and the below explanations are provided by way of example only, without limitation.

### INTRODUCTION

In particular, at least the above problem is solved by a system for treatment of patient tissue by delivery of high-voltage pulses comprising an ablation catheter, a measurement unit and an electronic control unit, whereby the catheter comprises a catheter shaft and an ablation portion being arranged at a distal end of the catheter shaft with a plurality of electrodes accommodated along the ablation portion, wherein each of the plurality of electrodes is electrically connected to a measurement unit through the catheter shaft, wherein the measurement unit is configured to perform measurements using an energy source thereby determining measurement values, in particular bipolar impedance measurement values of electrode pairs of a subgroup of the plurality of electrodes and/or quasi-unipolar impedance measurement values of a subgroup of the plurality of electrodes and/or current measurement values of a subgroup of the plurality of electrodes, wherein said subgroup is formed by all or a part of the plurality of electrodes, respectively, whereby the impedance and/or current measurement values may be determined as response to an alternating voltage and/or at least one voltage pulse, wherein the electronic control unit (ECU) may be arranged proximal to or at the proximal end of the catheter, wherein the measurement unit may be connected to or integrated within the ECU, wherein the ECU is configured to receive and analyze said measurement values provided by the measurement unit and determine arcing risk (AR) and/or a contact uniformity (CU) and/or an impedance uniformity (IU) indexes based on measurement values.

The arcing risk and/or the contact uniformity indexes may be based on the impedance measurement values and/or impedance for said electrodes. The impedance uniformity indexes may be based on the current measurements.

The above ablation catheter includes hardware and a respective algorithm to reliably indicate the risk for arcing and contact uniformity of electrodes. CU is important as it provides an immediate understanding to operating HCPs about the tissue contact uniformity over all active electrodes.

Within the frame of this application, the phrase “subgroup of electrodes” is understood as a pre-defined group of electrodes of the plurality of electrodes of the ablation portion of the ablation catheter which may be formed by all electrodes of the ablation portion or a real part of the electrodes of the ablation portion. For example, the ablation portion may comprise ablation electrodes and mapping electrodes as described below. In this example, the subgroup of electrodes may contain the ablation electrodes, only, but not the mapping electrodes (i.e. electrodes exclusively used for mapping).

In accordance with an embodiment, the system is configured for delivering pulsed-field ablating (PFA) energy to the patient's tissue by a health care practitioner (HCP), for example to the atrial or ventricular tissue of the patient's heart, via electrodes (also referred to as ablation electrodes) located along the ablation portion at the distal end of the ablation catheter. In other words, the system may be configured for carrying out PFA. In particular, the ablation catheter may be used to provide cardiac catheter ablation to treat a variety of cardiac arrhythmias including AF. For example, the system may comprise a multi-channel PF energy generator and the ablation catheter may be configured for being connected to a multi-channel PF energy generator which is configured for delivering PF energy. The waveform of said PF energy generator is conceived so that it, in conjunction with catheter loop or spiral design, achieves intended therapeutic effect while minimizing or reducing chances of ionization and the intended impedance and current measurements as indicated above. The inventive catheter may also be used for different type of tissue, for example veins, lungs, liver, kidneys. It may be used for pulmonary vein isolation (PVI), persistent atrial fibrillation ablation, ventricular tachycardiac ablation and other ablation procedures.

The inventive ablation catheter using PFA is intended to render tissues non-viable by irreversible electroporation (IRE). During IRE the electric field provided by the electrodes accommodated at the neighboring loop sections creates pores in cardiac cell membranes.

When the number of pores and their sizes are sufficiently great IRE occurs and the cell programs itself to die. For that neighboring loop sections of the ablation portion form a so-called ablation area.

The system comprises a measurement unit and an electronic control unit (ECU). The system may further comprise a multi-channel PF energy generator (further referred as PF generator) as an energy source. The measurement unit and the ECU may be integrated in the PF generator. The electronic control unit (ECU) may also be configured for controlling ablation procedure, in particular the PF generator, and receiving, processing and analyzing measurement values. The ECU comprises a microprocessor, computer or the like and is regarded as a functional unit of the system that interprets and executes instructions comprising an instruction control unit and an arithmetic and logic unit.

The catheter shaft may comprise a handle at its proximal end. Each electrode of the plurality of electrodes at the ablation portion is electrically connected via one electrical conductor to the PF generator provided at the proximal end of the catheter shaft. In an alternative embodiment, the measurement unit and/or the ECU may be at least partially integrated in the handle.

The PF generator, ECU and/or the measurement unit may be connected to or may comprise a memory module for storing data, e.g. measurement values or determined data calculated by the ECU from these measurement values. The memory module of the PF generator, the ECU and/or the measurement unit may include any volatile, non-volatile, magnetic, electrical media, or otherwise such as a random access memory (RAM), read-only memory (ROM), non-volatile RAM (NVRAM), electrically-erasable programmable ROM (EEPROM), flash memory, or any other memory storage type. The ECU may further be connected to a (graphical) user interface (GUI), e.g. for the HCP, in order to receive data input and/or display the determined AR indexes, CU value, impedance values and/or IU value.

In another embodiment, there are two electrical conductors provided at the proximal end and the middle section of the catheter shaft. At the proximal end, the first electrical conductor is connected to the first group of electrodes and the second electrical conductor is connected to the second group of electrodes in order to reduce the diameter of the catheter shaft. One electrode consists of electrically conducting material, for example, at least one of gold and a platinum/iridium alloy and/or may have a length along the respective ablation portion section of 1 mm to 10 mm, preferably 3 mm to 5 mm. The catheter shaft size may be compatible with a 7 F to 14 F ID sheath, preferable with an 8.5 F ID sheath. The width between adjoining electrodes along the respective loop section may be chosen between 1 mm and 10 mm, preferably 3-6 mm, in order to provide a contiguous ablated area at the patient's tissue.

In one embodiment, the length of the ablation electrodes may be in the range 3-5 mm. In one embodiment, the ablation electrodes may be sleeve-shaped or tubular. For example, a diameter of such a sleeve-shaped or tubular ablation electrode may be in the range of 2-2.5 mm. Further, as mentioned above, a length of the sleeve-shaped or tubular ablation electrode may be in the range of 1-10 mm, for example 3-5 mm. Alternatively, a split electrode design may be used. In this embodiment, two electrodes in form of half-shells separated by a gap are arranged at the inner side (facing the body lumen) and the outer side (facing the tissue) of the catheter. The gap may be 0.2-1 mm wide, preferably 0.5 mm wide. Alternatively, electrodes may be solid but coated with insulating material on the inner side facing blood (the body lumen). Parylene, Polyimide or Teflon are examples of a suitable coating. The coating material should be an electrical insulator with high dielectric strength, in excess of 200 kV/mm.

Each of the electrodes is electrically connected to the electronic control unit (ECU), wherein the connection may be provided via the PF generator to pair each two of at least two electrodes of the subgroup of electrodes in a pre-defined manner in order to operate the electrodes in a bipolar arrangement. If there are more than two electrodes, for example 16 electrodes, e.g. each two electrodes which are accommodated adjacent along the ablation portion may be paired (mode along the ablation portion) or each two electrodes which are accommodated adjacent across two neighboring loop sections of the ablation portion (see description of loop structure below, mode across loop sections) may be paired to be operated in a bipolar arrangement. Accordingly, 8 pairs may be formed from 16 electrodes in both modes. The pairing may be switched between the two modes. Further, the pairing may be switched to another pair of electrodes, for example along the loop sections. For pairing, the electrodes may be connected to a switch unit, wherein the switch unit is connected and controlled by the ECU. The ECU may further be adapted to switch into the below-mentioned ablation mode and mapping mode for each electrode, respectively. The switch unit realizes the pairing along the loop sections and, if applicable, the switching between the modes according to the control signals of the ECU.

In one embodiment, the measurement unit may be separate from or integrated within the ECU, wherein the measurement unit is configured to provide an activation signal in form of an AC voltage signal, AC current signal or at least one voltage pulse. In the case that the measurement unit is separate from the ECU, the measurement unit is electrically connected to the ECU.

In one embodiment, the measurement unit is configured to determine at least one current measurement value for each of the subgroup of the plurality of electrodes by measuring the respective current value of one or several of rectangular, sinusoidal, tooth or similar shaped voltage pulses, wherein one impedance value is determined from said determined current measurement values for each of the subgroup of electrodes. In this embodiment, the current measurement value is determined by a quasi-unipolar arrangement, wherein one of the electrodes of the ablation portion forms the reference electrode. In other words, the impedance value for an electrode is determined by using this electrode as reference electrode and measuring the current values in response to the voltage pulse at least one electrode of the subgroup of electrodes, in particular the current values of all or selected electrodes of different polarity of the subgroup compared to the reference electrode. For each of the electrodes the peak current is determined, wherein the peak voltage may be chosen between 1V and 1 kV, in particular between 10V and 700V, in particular between 100V and 500V. Each pulse comprises a positive and a negative half-wave having a rectangular, sinusoidal, tooth or similar shaped voltage pulse. The ECU analyzes the measurement values received from the measurement unit and determines from the peak voltage and the measured peak current value the impedance value for each electrode separately, wherein a mean value is determined for each electrode if the peak current is determined from more than one voltage pulse for each electrode. The frequency of the voltage pulse is, for example, 500 kHz. The determined impedances for each electrode may be presented to the HCP, for example, by means of a bar diagram, wherein the height of the bar represents the impedance value of the respective electrode. In one embodiment, the impedances for different positions of the ablation catheter with regard to the tissue may be presented for each electrode side by side. Further, a mean impedance value may be determined from all electrodes of the subgroup or a group of the subgroup, for example the proximal group and distal group. If the impedance value differs from the respective mean impedance value by a pre-defined percentage, the respective bar may be colored or otherwise highlighted thereby indicating to the HCP that the respective electrode is short circuited or malfunctional.

In one embodiment, the ECU is configured to determine an impedance uniformity (IU) value of the electrodes, wherein IU*=1−½*(σ({Z.sub.n})/μ({Z.sub.n})), wherein Z.sub.n is the impedance of the electrode n and σ({Z.sub.n}) is the standard deviation of the impedance values of all electrodes of the subgroup and μ({Z.sub.n}) is the mean value of the impedance values of all electrodes of the subgroup. In an alternative embodiment, the mean value and the standard deviation of two groups of the subgroup of electrodes are calculated separately, such that

$$IU = 1 - 12 ( σ ( \{ Z d \} ) μ ( \{ Z d \} ) + σ ( \{ Z p \} ) μ ( \{ Z p \} ) ) ,$$

wherein σ({Z.sub.d,p}) is the standard deviation and μ({Z.sub.d,p}) the mean value of the respective of the impedances of the electrodes of the respective group d/p, determined by the current measurement for each electrode as indicated above. The impedance values used for IU/IU*-determination are the impedances determined by current measurements as indicated above. The IU/IU* value provides a measure that indicates whether all catheter electrodes have equal contact with their surroundings or not, for example with the targeted tissue. To account for design differences, it can be evaluated by the range of impedance values for different groups of electrodes of the subgroups, for example, the distal group (d) and the proximal group (p). An IU/IU* value close to 1 indicates that all electrodes have identical contact. If the IU/IU* is low, for example, between 0.8 and 0.9, the contact uniformity is regarded mediocre, whereas IU/IU* below 0.8 is identified as bad contact uniformity. In this case, the HCP needs to change the position of the ablation catheter, in particular the position of the ablation portion with regard to the targeted tissue in order to increase contact uniformity. Such situation must be recognized prior high energy delivery during the patient's treatment.

The impedance uniformity explained above has the same function as the CU. Both parameters are important as they provide an immediate understanding to operating HCPs about the tissue contact uniformity over all ablation electrodes.

The AR index indicates the risk for arcing for a particular electrode pair. This parameter is essential for ablation catheters which operate at bipolar PFA and where electrode distances among each other can change due to manipulation, in particular where electrodes of different polarity come close. The AR index predicts opposing electrodes well and indicates their proximity. Furthermore, a strong correlation between the AR index and an actual arcing threshold for a given PF energy is observed. Additionally, the AR index may be displayed to the HCP prior treatment in an easy way. The AR index provides a more robust approach compared to a simple impedance measurement and avoids cross-sensitivity.

In one embodiment, the ECU is configured to determine the AR index for a particular electrode pair x,y from the bipolar impedance measurement values of the particular electrode pair x,y from the subgroup of electrodes scaled by the minimum of bipolar impedance measurement values of the respective electrodes with their adjoining electrodes of the subgroup. The bipolar impedance of a particular electrode pair x,y may be measured by applying an AC voltage for example at 500 kHz between the electrodes x,y. In this embodiment adjoining electrodes along the ablation portion of the subgroup have consecutive numbering. This means that adjoining electrodes identifiable with a consecutive numbering show their risk for arcing if one uses the AR index, wherein the AR index may be calculated using the bipolar impedance measurement values Z.sub.x,y for the respective pair of electrodes x,y. For a non-uniform contact of all electrodes (Z.sub.x,x+1 of the electrodes can be quite different, thus this should be considered as the general case), each non-adjoining measurement will get an RV-value, where x and y are the index of the electrodes, so that

$$AR x , y = 1 - Z x , y min ( Z x - 1 , x , Z x , x + 1 , Z y - 1 , y , Z y , y + 1 ) .$$

The AR index of a particular electrode pair x, y of the subgroup is defined between 0 (low AR risk) and 1 (high AR risk). If the calculated number becomes negative, the AR.sub.x,y is set to zero. Z.sub.x−1,x Z.sub.x,x+1 refer to bipolar impedances of the electrode x and its adjoining electrodes, whereas Z.sub.y−1,y Z.sub.y,y+1 refer to bipolar impedances of the electrode y and its adjoining electrodes. The expression min ( . . . ) defines the minimum of the impedance values indicated in parenthesis. It was shown in experiments that an AR index greater than 0.25 or, in another embodiment, greater than 0.15, causes arcing for the intended PF energy, wherein the AR index not only considers electrodes located in close and direct proximity but also electrodes which are close at their edges as these configuration may have an arcing threshold (voltage at which arcing occurs) that is lower than the maximum amplitude used for treatment for the particular ablation catheter.

In one embodiment, an AR index threshold, for example 0.15, may be defined which denotes a threshold from which arcing is observed for a particular catheter type and pulse protocol. I.e. if an AR index equal to or greater than the AR index threshold is observed, arcing is most likely noticed for the respective electrode pair. The threshold value may be defined such that it always addresses edge-edge positions. With staggered electrodes the value remains the same, but the likelihood of getting high AR values will be dramatically minimized. Staggered electrodes should be understood as an arrangement of the electrodes at the distal end of the ablation catheter whereby electrodes at the same polarity are geometrically closest. In that case the threshold may be determined more conservatively and uses even lower numbers. It is important to note that this relationship is only valid for the chosen pulse protocol as this influences the arcing threshold. Moreover, modifications of the catheter design, such as electrode length and spacing, also influence the relationship of arcing threshold and AR index. In the case that electrode lengths are not constant (e.g. use of 4 mm and 3 mm electrodes), one may add weights to the algorithm to take the different surface areas (which change the magnitude of Z) into account.

The AR index of the electrodes of the pairs x,y of the subgroup may be displayed to the HCP by means of a two-dimensional matrix (rows and lines referring to the electrode number and each intersection referring to the respective electrode pair x,y) highlighting the adjoining and/or opposing electrodes (e.g. electrodes of opposing loops of the ablation section), for example. Alternatively, the electrode pairs exhibiting a higher AR index may be highlighted at a respective visual representation of the ablation section and its electrodes located along this ablation section. The HCP may easily recognize from such visualization whether a repositioning of the catheter with regard to the patient's tissue is necessary.

In one embodiment, the ECU is configured to determine an overall risk for arcing for all electrodes of the subgroup AR.sub.max based on a maximum of the AR index of all electrode pairs of the subgroup, for example determined as indicated above. The overall risk for arcing refers to a particular position of the ablation catheter with regard to the patient's tissue to be treated.

$$AR . sub . max = max ( AR . sub . x , y )$$

The CU value is a parameter that indicates the quality of contact of all electrodes of the pre-defined subgroup, wherein the CU value refers to a specific position of the ablation catheter with regard to the patient's tissue to be treated. For a reliable ablation treatment result, the contact of the electrodes of the subgroup should be uniform over the whole subgroup.

In one embodiment, the ECU is configured to determine the CU value for the subgroup of electrodes based on the standard deviation of the bipolar impedance measurement values of the pairs of adjoining electrodes of said subgroup or based on the minimum and the maximum of the bipolar impedance measurement values of the pairs of adjoining electrodes of said subgroup. The bipolar impedance of a particular electrode pair x,y may be measured by applying an AC voltage for example at 500 kHz between the electrodes x,y. In one embodiment, the standard deviation of the bipolar impedance measurement values is compared with the mean value of these measurement values. For example,

$$CU = 1 - σ ( \{ Z n , n + 1 \} ) μ ( \{ Z n , n + 1 \} ) ,$$

wherein μ({Z.sub.n,m+1}) is the mean value of two adjoining electrodes of the subgroup and σ({Z.sub.n,n+1}) is the standard deviation of these electrodes. It was observed that good contact uniformity is realized if a CU value of about 1 (i.e. small standard deviation) is determined and a very heterogenous contact is detected if the CU value is close to zero. It is noted, that a good contact uniformity exists, too, if there are all electrodes of the subgroup without contact, e.g. floating in the blood pool. For example, a very uniform contact is given by CU values greater than 0.95. Mediocre CU is observed at a CU value of less than 0.90.

In an alternative embodiment, the CU value may be determined by the ECU using following calculation rule:

$${CU}^{′} = 1 - σ ( \{ Z n , n + 1 \} ) μ ( \{ Z n , n + 1 \} )$$

This embodiment is basically identical to the above calculation rule for CU but provides a more “spread out” of the CU data. A mediocre CU′ is observed for CU′=0.72 and a bad CU for a CU′ value of 0.67 and less.

In an alternative embodiment, the CU value may be determined by the ECU using the following calculation rule:

$${CU}^{″} = min ( 1 - max ( \{ Z n , n + 1 \} ) - μ ( \{ Z n , n + 1 \} ) μ ( \{ Z n , n + 1 \} ) , 1 - μ ( \{ Z n , n + 1 \} ) - min ( \{ Z n , n + 1 \} ) μ ( \{ Z n , n + 1 \} ) ,$$

wherein min and max refer to the respective minimum and maximum value, respectively. This embodiment puts stronger emphasis on outliers as it compares the maximum and minimum values of Z to the average. It was observed that a CU″ value of 0.83 refers to a mediocre CU, whereas a CU″ value of 0.79 or less has a bad CU.

In an alternative embodiment, the CU value may be determined by the ECU using the following calculation rule:

$${CU}^{″′} = 1 - max ( \{ Z n , n + 1 \} ) - min ( \{ Z n , n + 1 \} ) μ ( \{ Z n , n + 1 \} ) .$$

This embodiment has the same tendency as the above calculation rule for CU″ and puts even more emphasis on outliers. It was observed that a CU′″ value of 0.68 refers to a mediocre CU, whereas a CU″ value of 0.60 or less has a bad CU.

In an alternative embodiment, the CU value may be determined by the ECU using the following calculation rule:

$${CU}^{″″} = 1 - max ( \{ Z n , n + 1 \} ) - min ( \{ Z n , n + 1 \} ) max ( \{ Z n , n + 1 \} ) .$$

This embodiment has the same tendency as the above calculation rule for CU″ and CU′″. Here the scaling is not relative to average contact but relative to the best contact. It was observed that a CU″″ value of 0.72 refers to a mediocre CU, whereas a CU″ value of 0.67 or less has a bad CU. In some embodiments it might be advantageous to combine two or more of the described methods for calculating the contact uniformity CU.

In another embodiment, the ECU is configured to determine the CU value for the subgroup of electrodes based on the standard deviation of quasi-unipolar impedance measurement values of all electrodes of said subgroup or based on the minimum and the maximum of the quasi-unipolar impedance measurement values of all electrodes said subgroup. The quasi-unipolar impedance measurement values are determined by measuring one electrode against all electrodes of opposing polarity (e.g. electrode 1 versus all even electrodes). In order to determine the quasi-unipolar impedance value for all electrodes, the impedance of each electrode of the even group of the subgroup is measured against one pre-defined odd electrode with the number and the impedance of each electrode of the odd group of the subgroup is measured against one pre-defined even electrode. Accordingly, there is one impedance measurement value for each electrode n of the subgroup, as n is either odd or even. The CU values are determined as described in the above calculation rules for CU, CU′, CU″, CU′″, CU″″, wherein Z.sub.n,n+1 is replaced by the impedances of the respective electrode Z.sub.n. The above explanations with regard to bipolar impedance measurements for determination of CU value apply for the quasi-unipolar impedance value, as well. However, if one even and one odd electrode are in close proximity (or in worst case) in contact, all CU value determination is strongly influenced by this condition. Then, the measurement exhibits a bipolar character.

In an alternative to the embodiment as described above, the quasi-unipolar impedance measurement values are determined by measuring one electrode against a selected group of electrodes of opposing polarity (e.g. electrode 1 versus a selected group of even electrodes). In this embodiment, the electrodes of opposing polarity are grouped in at least two groups based on the distance to the one electrode. For each electrode at least two groups of opposing polarities may be defined. A first group of the at least two groups may comprise the electrodes of opposing polarity near-by to the one electrode. A second group of the at least two groups may comprise the electrodes of opposing polarity far away to the one electrode. In this embodiment the ECU is configured to determine the CU value for the subgroup of electrodes based on the standard deviation of quasi-unipolar impedance measurement values of all electrodes of said subgroup or based on the minimum and the maximum of the quasi-unipolar impedance measurement values of all electrodes said subgroup, whereby the quasi-unipolar impedance values are determined by measuring one electrode against all electrodes of the second group. Quasi-unipolar impedance measurements based on the first group comprising the near-by electrodes are not considered in this embodiment. Thereby a bipolar character of the quasi-unipolar measurements is avoided.

With regard to above measurements in one embodiment, the measurement unit is configured such that the frequency for determination of quasi-unipolar or bipolar impedance measurement values of the subgroup of electrodes is between 1 kHz and 1 MHz and/or such that the voltage amplitude of the pulses is between 1V and 1 kV, in particular between 10V and 700V, in particular between 100V and 500V.

In another embodiment, it is considered that impedance measurements are also sensitive to electrode design (e.g. length, radius, spacing). These parameters can vary on the catheter (e.g. distal electrodes shorter than proximal electrodes). Since the designs are known, the algorithm realized by the ECU accounts for the differences by an additional scaling factor. For instance, measurements between 4 mm and 6 mm electrodes are treated differently than for 6 mm to 8 mm electrodes (comparably lower impedance expected). So, impedance measurements may be scaled using that information. The information on catheter design may also be used to simplify the algorithm. If electrodes cannot touch or come close by design (e.g. they are on opposite sides of one spline) then this reduces the number of measurements needed for the algorithm. If it's desired to have the generator working with unknown catheters, a pretesting method may be implemented. This could be realized by asking the HCP to place the catheter in the blood (no wall contact). By pretesting the catheter measures impedance of pairs of adjoining electrodes and the difference in values can be ascribed to the catheter design. Furthermore, this possibly allows for a reduction of impedance measurements as this precheck may already identify electrodes that are far apart and will not touch even if the catheter is compressed and/or twisted. Alternatively, the measurements may be performed constantly and the lowest values (contact increases impedance) will be taken for the scaling process.

For providing the measurements the system is configured to select electrodes (e.g. using a multiplexer). If needed, accuracy can be increased by averaging multiple measurements and/or perform measurements synced to the QRS complex of the patient's heart in order to reduce artefacts from the beating heart. However, the advantage of improved data quality needs to be balanced with the inherent prolongation of the measurement duration.

In one embodiment, beyond measurements the catheter also allows to select electrode groups to apply pulsed energy. This feature may be achieved, as an example, by interaction with a user interface located on the catheter front panel. The HCP may proactively select groups of therapy-providing electrodes. The selection may be made such that electrodes with a high AR index or with an uneven CU value are placed in different therapy groups.

According to another aspect of the present invention, at least the above problem is solved by a method for assessment of positions and/or configuration of a plurality of electrodes of an ablation catheter for treatment of patient tissue by delivery of high-voltage pulses comprising a catheter shaft and an ablation portion, wherein the ablation portion is arranged at a distal end of the catheter shaft with the plurality of electrodes accommodated along the ablation portion, wherein each of the plurality of electrodes is electrically connected to a measurement unit through the catheter shaft, wherein the measurement unit performs measurements using an energy source thereby determining measurement values, in particular bipolar impedance measurement values of electrode pairs of a subgroup of the plurality of electrodes and/or quasi-unipolar impedance measurement values of a subgroup of the plurality of electrodes and/or current measurement values of a subgroup of the plurality of electrodes, wherein the subgroup is formed by all electrodes or a part of the plurality of electrodes, respectively, whereby the impedance and/or current measurement values may be determined as response to an alternating voltage and/or at least one voltage pulse, wherein an electronic control unit (ECU, 70) may be arranged proximal to or at the proximal end of the catheter, wherein the measurement unit may be connected to or integrated within the ECU, wherein said measurement values are transmitted to the ECU which receives and analyzes said measurement values as well as determines arcing risk (AR) and/or a contact uniformity (CU) and/or an impedance uniformity (IU) indexes based on said measurement values.

The arcing risk and/or the contact uniformity indexes may be based on the impedance measurement values and/or impedance for said electrodes. The impedance uniformity indexes may be based on the current measurements.

The above method (or algorithm) was already described with regard to the system above. It is therefore referred to the above explanation. The above method may be provided prior a PFA treatment using the electrodes of the ablation portion of the same system or in between two cycles of such treatment.

In one embodiment of the method, the measurement unit determines at least one current measurement value for each of the subgroup of the plurality of electrodes by measuring the respective current value of one or several of rectangular, sinusoidal, tooth or similar shaped voltage pulses, wherein one impedance value is determined from said determined current measurement values for each electrode of the subgroup of electrodes.

In one embodiment of the method, the ECU determines an impedance uniformity (IU) of two groups d,p of the subgroup of electrodes, wherein

$$IU = 1 - 12 ( σ ( \{ Z d \} ) μ ( \{ Z d \} ) + σ ( \{ Z p \} ) ) \cdot Math ,$$

wherein σ({Z.sub.d.p}) is the standard deviation and μ({Z.sub.d,p}) the mean value of the determined impedances of the electrodes of the respective group.

In one embodiment of the method, the ECU determines the AR index for a particular electrode pair x,y from the bipolar impedance measurement values of the particular electrode pair x,y from the subgroup of electrodes scaled by the minimum of bipolar impedance measurement values of the respective electrodes with their adjoining electrodes of the subgroup.

In one embodiment of the method, the ECU determines the CU value for the subgroup of electrodes based on the standard deviation of the bipolar impedance measurement values of the pairs of adjoining electrodes of said subgroup or based on the minimum and the maximum of the bipolar impedance measurement values of the pairs adjoining electrodes of said subgroup and/or determines the CU value for the subgroup of electrodes based on the standard deviation of the quasi-unipolar impedance measurement values of all electrodes of said subgroup or based on the minimum and the maximum of the quasi-unipolar impedance measurement values of all electrodes of said subgroup.

In one embodiment of the method, the ECU determines an overall risk for arcing for all electrodes of the subgroup based on a maximum of the AR index of all electrode pairs of the subgroup.

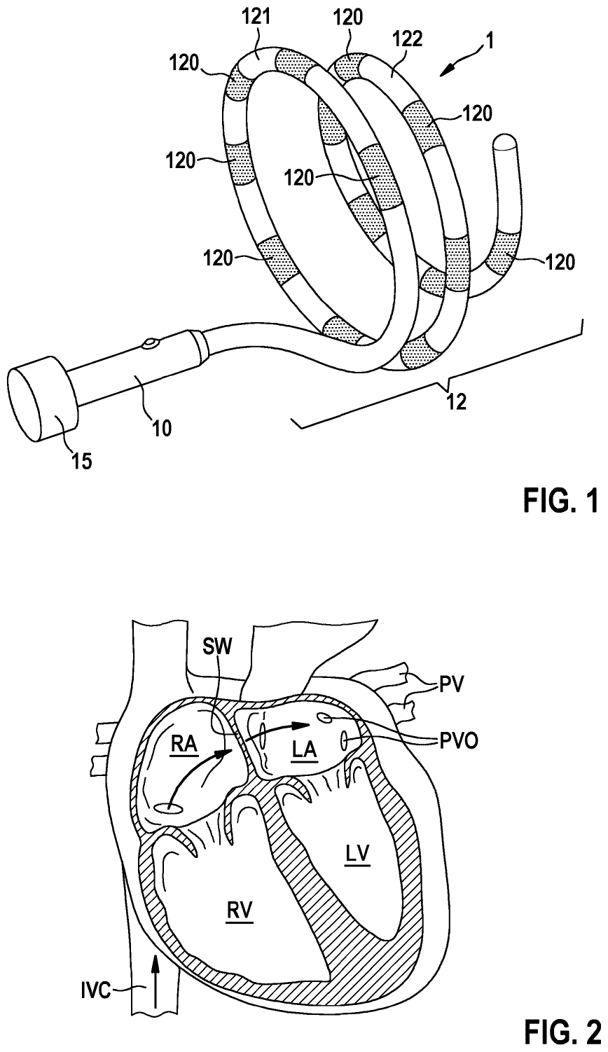

In one embodiment, the ablation portion comprises at least one loop section, for example at least two loop sections forming a three-dimensional spiral. The at least two loop sections may be arranged as a continuous or discontinuous spiral. In this case, the beginning and the end of each loop section could be arranged either in the same or in a different plane with respect to the central axis of the three-dimensional spiral. In addition, the at least two loop sections itself could be arranged either in the same or in a different plane with respect to the central axis of the three-dimensional spiral. An example of at least two loop sections forming a continuous spiral is shown in FIG. 1, whereby the beginning and the end of each loop section is arranged in a different plane with respect to the central axis of the three-dimensional axis and whereby the at least two loops are arranged in different planes with respect to the three-dimensional axis.

In one embodiment, the diameters of two neighboring loop sections increase into the direction of the distal end of ablation portion forming a plunger type ablation catheter. The plunger type ablation catheter may be used for ablation in the ventricles or in the atrial area of the posterior left atrium. Alternatively, the diameters of two neighboring loop section decrease into the direction of the distal end of the ablation portion forming a corkscrew type ablation catheter. The corkscrew type ablation catheter may be used for ablation in the area of the atrial end of the PV. The diameters of loop sections may be, for example, between 10 mm and 40 mm. More specifically, if used in the left atrium, the widest loop section may have a diameter between 20-35 mm, preferably between 25-32 mm. The smallest diameter can be 12-22 mm, preferably 15-20 mm. The diameter is measured from both inner surfaces of opposite loop sections. For both areas, the form of the ablation portion is adapted to the specific form of the respective area to be ablated.

It is also within the scope of the present invention that the ablation portion may comprise a plurality of separate mapping electrodes, the mapping electrodes being configured for receiving electrical signals, e.g. electrical or biopotential, from ventricular, vascular or atrial tissue. Alternatively, the electrodes used for ablation in the ablation mode may be used for mapping, namely receiving electrical biosignals, e.g. acquiring electrical or biopotential, from ventricular, vascular or atrial tissue. During ablation these electrodes are in the ablation mode. This may enable mapping and ablation with a single ablation catheter for PVI as well as ablating some non-PV triggers for AF patients.

For example, in an embodiment, an additional loop section of the plurality of loop sections may exhibit a plurality of mapping electrodes (electrodes exclusively used for mapping). Additionally or alternatively, mapping electrodes may also be arranged—in addition to the ablation electrodes—on one or both of the two neighboring loop sections. A plurality of mapping electrodes may also be incorporated distal to the plurality of ablation electrodes, or medially within two ablation electrodes, e.g. between two ablation electrodes (along the respective loop section). Furthermore, the third loop section may comprise ablation electrodes in addition to or instead of the mapping electrodes.

In one embodiment, the ablation portion, and in particular the loop sections, may comprise a shape memory material. Preferably, the shape memory material is a super-elastic material (such as a super-elastic alloy), which is to say that the material is elastic and has a shape memory property. For example, Nitinol is a biocompatible super-elastic alloy that is suitable for the present purpose. In one variant, the ablation portion, and in particular the loop sections, may comprise an inner support element, such as an inner support wire, having a shape memory or super-elastic property. The shape memory support wire may have various stiffness and cross-sectional shapes in different sections. The inner support structure maintains the architecture and design integrity of the ablation portion and extends along at least a section of the ablation portion. The inner support structure may be realized as a Nitinol wire (for example a round, rectangular, square wire with variable cross section or tapered). In addition, this support structure comprises insulated with material, for example Parylene, Polyimide, Teflon at the outer surface of the wire. Further, the wire of the ablation portion may have sections with different diameter or cross-sectional shape in order to provide different stiffness.

In an embodiment, the ablation catheter may further comprise a steerable delivery sheath. Thus, in operation, a position of the ablation portion may be easily adjusted at the target tissue until the contact of each ablation electrode is satisfied.

In one embodiment, the catheter shaft comprises at least two lumens separated by a material with a dielectric strength greater than a dielectric threshold suitable to withstand high-voltage PF pulses used with the above and below described system/catheter, for example with high-voltage PF pulses having an amplitude greater than 1 kV, greater than 2.5 kV or between 2.5 kV and 3.5 kV. Such material may be, for example, a polymer film, in particular a Polyimide film (e.g. Kapton® film) provided in form of tubing or a layer received by dipping. It has a dielectric strength of 160 kV/mm. The thickness of the polymer film (Polyimide layer) may be chosen in the range of 0.012 mm to 0.125 mm, for example. In this embodiment, the first lumen of the at least two lumens is configured to retain at least two electrical conductors which are connected with electrodes providing the same first polarity and wherein the second lumen of the at last two lumens different from the first lumen is configured to retain at least two electrical conductors which are connected with electrodes providing the same second polarity different from the first polarity. This embodiment allows to reduce the diameter of the catheter shaft as the isolation of each electrical conductor is not necessary and to provide necessary safety with regard to arcing at the same time.

In one embodiment, the catheter shaft may have an overall length greater than 1 m from the handle to the distal tip of the ablation portion.

In one embodiment, at least two of the plurality of electrodes of the ablation portion are adapted to deliver high voltage unipolar PF energy or bipolar PF energy or a combination of unipolar and bipolar PF energy as described below. A schematic example of an applicable waveform is shown in FIG. 13. Such waveforms, in combination with the loop structures described above, ensure one-shot application of electrical fields that are high and long enough to generate therapeutic effects capable of creating moats of conduction block, yet lower and shorter than ionization thresholds so to avoid arcing. The PFA pulses can be delivered gated by the QRS complex of the cardiac cycle. Alternatively, when ablation targets regions remote from ventricles, PFA pulses may be delivered asynchronously, without QRS gating. The electronic control unit is adapted to switch between unipolar PF energy and bipolar PF energy supply mode.

In another embodiment, the distal tip of the ablation portion is connected with steering wires or center wire which may be manipulated from a handle element provided at the proximal end of the catheter shaft. Accordingly, the center wire may be connected to an actuation mechanism within the handle element. Along the ablation portion, the center wire approximately run along a longitudinal axis of the catheter shaft. A steering plate, steering ring, or other known steering structures may be placed at the distal end of the catheter shaft, which connects to the distal spiral, or multiple loop, ablation section. The center wire connects to said steering structure. The center wire may be manipulated such that a longitudinal length of the ablation portion (i.e. its length along the longitudinal axis of the three-dimensional spiral/multiple loop structure) or the loop sections may be steered towards tissue targets, according to the therapeutic needs.

In one embodiment, the electrodes are distributed along the at least two loops in a way, that the angular separation between the most distal and the most proximal electrode is at least 360°. The angular separation is determined by the angle between the most distal electrode, the catheter axis and the most proximal electrode. Furthermore, electrodes may be distributed along the at least two loops in a way, that the longitudinal separation between the most distal and the most proximal electrode is at least 5 mm. The longitudinal separation is understood as distance along the catheter axis between the most distal electrode and the most proximal electrode.

The above method is, for example, realized as a computer program (to be executed within the system, in particular within its ECU) which is a combination of above and below specified (computer) instructions and data definitions that enable computer hardware or a communication system to perform computational or control functions and/or operations, or which is a syntactic unit that conforms to the rules of a particular programming language and that is composed of declarations and statements or instructions needed for an above and below specified function, task, or problem solution.

Furthermore, a computer program product is disclosed comprising instructions which, when executed by a processor, for example a processor of the ECU, cause the processor to perform the steps of the above defined method. Accordingly, a computer readable data carrier storing such computer program product is described. The computer program product may be a software routine, e.g. related to hardware support means within the processor of the ECU.

In an alternative embodiment, one aspect relates to a generator for a substantially charge-balanced pulse for application onto at least one pair of therapeutic electrodes. The medical device may be a catheter with one or more electrodes. If the catheter carries only one electrode, a reference/return/grounding electrode could be used to close the output circuit. Alternatively, the medical device can be a hand-held surgical wand, or a surgical energy instrument. The generator may comprise a pulse-shaping output stage for coupling to the at least one pair of therapeutic electrodes. It may further comprise an internal pulse generator for applying an internal pulse to the pulse-shaping output stage. The pulse-shaping output stage may comprise a transformer and/or a capacitor system, the capacitor system comprising at least one capacitor, such that the internal pulse is transformed into a substantially charge-balanced pulse and applied to the at least one therapeutic electrode pair when coupled to said output stage.

The at least one therapeutic electrode pair may form an electrical load for the pulse-shaping output stage. The electrical load may be associated with various medical treatment modalities (e.g., a tissue treatment, e.g., a tissue ablation, a tissue stimulation, etc.) wherein the generator may be used to apply the substantially charge-balanced pulse to the treatment target. The electrical load may also be (partly) defined by a tissue and/or a path through the tissue. To illustrate an example, the least one pair of electrodes may facilitate a tissue treatment by contacting the respective tissue at a certain distance to each other. The two electrodes of a pair may thus form an electrical path through the tissue (or through the space defined by the two electrodes) wherein the electrical path may also be considered a part of the said electrical load. However, in another example, pulse-shaping output stage may apply the PFA energy to one or more single electrodes to facilitate a treatment by the medical device. Such application would be considered unipolar energy delivery, as it may involve a reference/return/grounding electrode that closes the electrical circuit. Alternatively, the PFA energy may be applied in bipolar mode to pair of electrodes located on one or more medical instruments.

A (substantially) charge-balanced output pulse may comprise that the net charge of the pulse is substantially zero. The charge-balanced pulse may for example comprise a (biphasic or multiphasic) voltage pulse having a net charge of substantially zero over a defined time window. The charge-balanced pulse may comprise a positive and a negative phase wherein the absolute value of the charge of the positive phase substantially equals the absolute value of the charge of the negative phase. The system according to this invention may also delivery pulses with multiple phases that are charge-balanced over the duration of the pulse. In another example, the charge-balanced pulse may also comprise a (biphasic or multiphasic) current pulse that has a net charge of substantially zero over the defined time window. The pulses may be high voltage pulses.

For example, a (substantially) charge-balanced pulse may comprise a (biphasic or multiphasic) voltage comprising a positive and a negative phases wherein the absolute value of the charge of the positive phases may be at least 90%, in particular at least 95%, at least 98%, at least 99% of the absolute value of the charge of the negative phases or vice versa. The charge-balanced pulse may comprise a (biphasic or multiphasic) voltage pulse having a net charge of less than ±5 μC, in particular less than ±2 μC, in particular ±1 μC over a defined time window. The defined time window may start when the positive (or negative) amplitude exceeds the measured noise level, in particular exceeds the noise level by a factor of 1.5, by a factor of 2, and may end when the negative (or positive) amplitude is below the 2 fold noise level, in particular below the 1.5 fold noise level, in particular below the noise level.

The generation of a reliable charge-balanced pulse may, for example, prevent a net charge injection into the stimulated tissue via the at least one electrode pair. Depending on the treated tissue and/or the type of treatment this may be highly beneficial. The charge balance may, for example, prevent the occurrence of electrolysis of blood, which may minimize an undesired gas generation within the organism. Generation of gas may cause microbubbling. In turn, microbubbling may lead to embolic events. For example, this may be highly beneficial when the treated tissue is within an organism such that it is surrounded by blood. The charge-balance enabled by the generator may also prevent undesired (skeletal) muscle contractions (e.g., which may be caused by direct or indirect stimulation of motor nerves). Further, the charge-balanced pulse may prevent an electrical arcing caused by ionization of the medium between electrodes of said at least one pair.

Notably, the (substantially) charge-balanced pulse may also enable a medical treatment requiring the application of a positive, as well as a negative charge. This may for example, be desired for a tissue ablation (e.g., an irreversible electroporation) the system may be configured for.

The coupling of the at least one electrode pair to the pulse-shaping output stage of the generator during an application of the charge-balanced pulse, as described herein, may be considered an active coupling of the electrode pair. For example, the medical device may comprise a plurality of electrode pairs which may be physically (e.g., mechanically) and in particular electrically connected to the pulse-shaping output stage of the generator. However, the coupling of the (substantially) charge-balanced pulse may be performed such as to selectively couple to all or only a subset of electrode pairs thereof. Such coupling may be enabled, for example, via a corresponding electrical circuit of the generator (e.g., comprising a switch) that activates all or a subset of desired electrode pairs. Such coupling that operatively couples the respective electrode pair will sometimes also be referred to as active coupling herein.

In an example, the internal pulse generator may be configured to apply an internal voltage and/or current pulse with a defined pulse duration to the pulse-shaping output stage. The internal pulse may comprise a rectangular pulse, however, also other types of internal pulses may be conceivable (e.g., a gaussian pulse, a cosine squared pulse, a Dirac pulse, a sinc pulse, etc.). The internal pulse may comprise only a positive voltage and/or only a positive current (or only a negative voltage and/or only a negative current).

The internal pulse may be applied as an input to the pulse-shaping output stage. The internal electrical pulse may then cause a (substantially) charge-balanced output pulse in the at least one coupled electrode pair. In an example, the pulse-shaping output stage of the generator may be part of a coupling circuit. Notably, the substantially charge-balanced pulse may be caused in the at least one electrode pair due to the dynamic response of the pulse-shaping output stage to the internal pulse. Notably, due to the internal pulse having a defined pulse duration the internal pulse can be considered a dynamic input to the pulse-shaping output stage. The pulse-shaping output stage may be configured to be adaptable to enable the desired dynamic response based on a given internal pulse and/or the characteristics of the electrical load that involves the at least one electrode pair. However, the characteristics of the internal pulse may also be adapted by the generator to enable the desired dynamic response of the present pulse-shaping output stage.

In an example, the pulse-shaping output stage may comprise a capacitor system with the at least one capacitor. The capacitor system may form a total capacitance of the pulse-shaping output stage. Moreover, the pulse-shaping output stage may be configured such that the total capacitance differentiates a voltage characteristic (dynamically) of the applied internal pulse. The resulting current and/or voltage characteristics in the at least one electrode pair may then result in the (substantially) charge-balanced pulse as a (dynamic) response. For example, the voltage differentiation by the total capacitance may cause an according electrical current characteristic in the at least one electrode pair. The electrical current characteristic may cause an according voltage applied to the at least one electrode pair which may correspondingly be in the form of the (substantially) charge-balanced pulse.

In an example of the generator, the at least one electrode pair may be coupled in series to the pulse-shaping output stage. For example, one electrode pair may be coupled in series with the capacitor system. The example may also comprise that one or more electrode pairs may be parallel to each other wherein said parallel circuit of electrode pairs is coupled in series to the pulse-shaping output stage. In this example, the coupling circuit may thus be considered to comprise a total capacitance in series with the total impedance of the one or more electrode pairs coupled to the capacitor system. The coupling circuit may thus form an RC-circuit. Notably, in that case, the coupling circuit may function (at least in part) as an RC differentiator circuit that transforms the internal pulse to the (substantially) charge-balanced pulse. To that regard, the voltage signal across the resistor of the RC differentiator circuit may be the voltage signal across the total impedance of the one or more electrode pairs, which forms said electrical load. Hence, the dynamic output of the RC differentiator circuit may be configured such that a (substantially) charge-balanced pulse is delivered to the one or more electrode pairs. The RC differentiator circuit may for example be constructed (or adapted) based on the characteristics of the internal pulse, the total capacitance and/or the total impedance of the one or more electrode pairs to enable the desired dynamic response.

In an example, the pulse-shaping output stage comprises the capacitor system and the generator may be configured such that at least two total capacitances of the capacitor system can be set. The total capacitance may thus be varied such that the overall electrical properties of the pulse-shaping output stage can be adapted, as well (e.g., to enable a desired dynamic response thereof). In an example, the capacitor system may comprise an electrical circuit comprising a plurality of capacitors arranged in one more circuit branches. In that case the generator and/or the capacitor system may be configured for switching one or more circuit branches on or off to set the at least two total capacitances. For example for electrical load values in the 15-25Ω range, desired capacitance values may be in the range of 0.2-0.5 μF. For loads in the 25-50Ω range, desired capacitance values may in the range of 0.1-0.2 μF. Yet for loads in the 50-150Ω range, desired capacitance values may be less than 0.1 μF. Ideally, if load values vary depending on the number of selected electrodes, the generator would adjust the capacitance value such that the equivalent time constant stays in the same range.

In another example, the generator may be configured to set at least three, preferably at least four, more preferably at least five, most preferably at least six total capacitances of the capacitor system.

In an example, the capacitor system may comprise a variable capacitor which may enable to set at least two total capacitances of the capacitor system.

In an example, the generator may be configured to set the total capacitance of the capacitor system based at least in part on an impedance of the electrical load associated with the at least one electrode pair. For example, the total capacitance may be set automatically based on the impedance of the electrical load associated with the at least one electrode pair. When one or more electrode pairs are (actively) coupled to the pulse-shaping output stage the impedance of the electrode load associated with the at least one electrode pair may comprise the total impedance of the one or more electrode pairs in the coupling circuit. The total capacitance may thus be set according to the total impedance present in the coupling circuit. For example, as outlined herein, the coupling circuit may comprise an RC circuit wherein the setting of the total capacitance enables to adjust the capacitance of the RC circuit. Also, when one electrode pair is coupled to the pulse-shaping output stage the total capacitance may only have to be set based on the impedance of the electrical load associate with the one electrode pair.

For example, the impedance of the electrical load associate with the at least one electrode pair and/or the total impedance may be measured by the generator (as described herein). The measurement may be performed based on a current and/or voltage measurement (as described herein).

In another example, the generator may be configured to receive the total impedance. The total impedance may thus be communicated to the generator such that the generator may adjust the total capacitance accordingly. For example, it may be predetermined that the active coupling of a first and second electrode pair of the medical device to the pulse-shaping output stage may result in a first total impedance wherein the active coupling of a third and fourth electrode pair may result in a second total impedance. In this example, the total impedance may, e.g., be inputted by an operator or may be received from an external device that has performed the impedance measurement.

In an example, the generator may be configured to set the total capacitance of the capacitor system based at least in part on a number of electrode pairs (actively) coupled to the pulse-shaping output stage. For example, the generator may be configured to receive the total number of electrode pairs coupled to the pulse-shaping output stage. In another example, the generator may be configured to determine the number of electrode pairs coupled to the pulse-shaping output stage. The number of electrode pairs (actively) coupled to the pulse-shaping output stage may indicate (or serve as benchmark) for the total impedance present in the coupling circuitry. Hence, the total capacitance may be set in a dependence to the total impedance present in the coupling circuit. The generator may be configured to automatically set the total capacitance of the capacitor system based on the (received or determined) number of electrode pairs. The automatic setting may be performed by an according computer program that can control the capacitor system wherein the computer program may be comprised by the generator and/or an external device. Also, the total capacitance may be manually set (e.g., by an operator) based on the number of coupled electrode pairs.

In an example, setting the total capacitance may not only be based on the number of coupled electrode pairs but also based on the medical device (or type of the medical device). The (total) impedance may not necessarily depend only on the number of coupled electrode pairs but also on the impedance of the electrical load associated with a respective electrode which may be different depending on the medical device.

In an example, the one or more electrode pairs may be parallel to each other when coupled to the pulse-shaping output stage. In this example, each impedance of an electrode pair may also be substantially the same, for example, due the design of the medical device or due to the uniformity of electrode-tissue contact. Hence, the number of electrode pairs may indicate the number of parallel branches wherein each branch comprises substantially the same impedance. A higher number of coupled electrode pairs may thus indicate a lower total impedance wherein a comparatively lower number of coupled electrode pairs may indicate a comparatively higher total impedance. Hence, the total capacitance may be accordingly adjusted. Notably, the above determination applies to both unipolar and bipolar energy applications. Unipolar energy applications require a reference/return/grounding electrode that closes the electrical circuit at the output of the generator.

For example, it may be conceivable that the total capacitance to be set by the generator is associated with a certain number of coupled electrode pairs. In that case, the generator may be configured such that when a first number of electrode pairs (of a particular medical device) is coupled to the generator, a first value of the total capacitance may be set. When a second number of electrode pairs (of the particular medical device) is coupled to the generator, a second value of the total capacitance may be set.

Notably, the electrode pairs being parallel to each other in the pulse-shaping output stage may be enabled by the generator. In an example, the generator may comprise an interface relay board for connecting the electrode pairs of the medical device to the pulse-shaping output stage. The interface relay board may comprise a (controllable, e.g., switchable) relay circuitry for coupling the electrode pairs to the pulse-shaping output stage in various ways. For example, the relay circuitry may enable to couple the electrode pairs in parallel to each other to the pulse-shaping output stage. However, in some examples, the interface relay board may also enable that the electrode pairs can be arbitrarily coupled to the pulse-shaping output stage (in series to each other, at least in part in series and at least in part in parallel, etc.). In an example of the relay board, it may comprise a first subgroup of relay channels which may be connected parallel to each other wherein each relay channel may be coupled with a first node of the pulse-shaping output stage. The relay board may comprise a second subgroup of relay channels which may be connected parallel to each other wherein each relay channel may be coupled with a second node of the pulse-shaping output stage.

In an example, the generator may be configured to set the total capacitance of the capacitor system such that a multiplication result of the total capacitance and an impedance (i.e. time constant) of the electrical load associated with the at least one electrode pair is within a predetermined range. For example, when one or more electrode pairs are coupled to the pulse-shaping output stage the impedance of the electrical load associate with the at least one electrode pair may comprise the total impedance of the one or more electrode pairs in the coupling circuit. In that case, the total capacitance and the total impedance may define an RC-circuit in the coupling circuit (as described herein). The multiplication result of the total capacitance with the total impedance may thus comprise the RC time constant τ (with τ=R·C, wherein R may comprise the total impedance, and C may comprise the total capacitance in that example). Hence, the generator may enable to set a desired RC time constant which may define the dynamic response of the coupling circuit. The RC time constant may be set such that a (substantially) charge-balanced pulse may be caused in the electrode pairs when the internal pulse is applied to the pulse-shaping output stage and the pulse-shaping output stage is electrically connected to the electrode pairs. This may be highly beneficial since without an according RC time constant the dynamic response may not necessarily lead to a (substantially) charge-balanced pulse. In addition, the waveform of the (substantially) charge-balanced pulse may be shaped depending on the set RC time constant. For example, it may be desirable to have a sufficient duration of the positive phase and a sufficient duration of the negative phase of the charge-balanced pulse which may be altered via the RC time constant. Furthermore, it may be desirable that both durations are substantially equal to create a (substantially) symmetric charge-balanced pulse for the medical application. Alternatively, the phase durations may be different so that to allow for the net charge to be balanced. Also, alternatively, the total capacitance value may be different for the positive and for the negative phases. This can be achieved by implementing a pulse-shaping output stage that detects the transition from positive to negative phases and drives the capacitor block to adjust its total capacitance. Accordingly, the adjustment of the RC time constant may enable to adapt the symmetry of the charge-balanced pulse since the RC time constant may function as one of the main performance parameters of the pulse-shaping output stage. Notably, some RC time constants may result in a charge-balanced pulse with two sharp positive and negative phases with a comparatively short duration, respectively, which may not always be desired depending on the medical treatment. Alternatively, one positive and one negative phase provides similar effects. Hence, the generator may be set to allow only the predetermined range of RC time constants such that extreme dynamic responses are avoided.

In another example, the generator may set the total capacitance such that a multiplication result of the total capacitance and an impedance of one electrode pair is within a predetermined range. For example, it may be necessary to adjust the RC time constant for a single electrode pair and not necessarily to the total impedance present in the coupling circuit (the whole circuit comprising the generator and all electrode pairs) to consider a desired outcome of its dynamic response. For example, this may be conceivable when the electrode pairs are coupled differently to the pulse-shaping stage than in a parallel assembly to each other. For example, unipolar vs. bipolar electrode configurations me require different time constants.

In an example, the pulse-shaping output stage may comprise the transformer such that the internal pulse is transformed into a substantially charge-balanced pulse and delivered to the at least one electrode pair when coupled to the pulse-shaping output stage. In this case the pulse-shaping output stage may comprise a part of the transformer (e.g., a coil or a winding of the transformer). This may ensure that the pulse-shaping output stage does not have a direct electrical connection (i.e. stage is not DC coupled) to the circuitry in which the high voltage internal pulse is generated. For example, a step-up transformer my be used. This may enable a galvanic isolation from the internal pulse generator. Additionally, it may boost the output voltage without placing additional voltage stress on the components of the internal pulse generator. Also in this case, the dynamic response of the coupling circuit may be adapted such that a (substantially charge-balanced pulse) is generated in the at least one electrode pair. To that regard, it may be conceivable that the inductivity of the transformer (e.g., of one of its coils) may be used in combination with the impedance of the at least one electrode pair to shape the dynamic response accordingly. For example, when using the transformer (e.g., without the capacitor system) the coupling circuit (the whole circuit comprising the generator and all electrode pairs) may comprise an RL-circuit whose dynamic response to the internal pulse can be adjusted (e.g., by adapting the inductivity L and/or the impedance R).

In an example, the pulse-shaping output stage comprises the transformer and the capacitor system, and wherein the generator is further configured such that the internal pulse is coupled from the internal pulse generator to the capacitor system via the transformer. Hence, the output of the internal pulse generator may be coupled to an input of the transformer, wherein an output of the transformer may be coupled to the capacitor system. The transformer may thus function as an intermediary element between the internal pulse generator and the capacitor system. This may enable a galvanic isolation of the capacitor system from the internal pulse generator. Moreover, a galvanic isolation from various other parts of the generator that are positioned at the input side of the transformer may also be enabled.

In an example, the transformer may be configured such that a voltage at the transformer output (which is coupled to the capacitor system) is higher than an applied voltage at the transformer input. The transformer may thus function as a step-up transformer that increases the input voltage by a predetermined amount at its output (e.g., depending on the relationship of the secondary windings to the primary windings of the transformer). The transformer may increase the input voltage by at least 10%, or 20% or by at least 30%, preferably at least 50%, more preferably at least 80%, most preferably at least 20-30%. This may be beneficial since the technical limitation of possible voltage amplitudes of the internal pulse generator may be overcome. For example, switching elements (e.g. FET transistors) of the internal pulse generator may be exposed to handling optimal current transients.

However, it may also be conceivable that the transformer may be configured such that the voltage at the transformer output (which is coupled to the capacitor system) is lower than the applied voltage at the transformer input.

In an example, the generator further comprises: means for measuring a voltage and/or a current of the at least one electrode pair when the substantially charge-balanced pulse is applied; means for determining an impedance of the at least one electrode pair based on the measurement of the voltage and/or the current. For example, the means for measuring the voltage and/or the current may be configured to measure the voltage across the at least one electrode pair (or the voltage across a plurality of parallel electrode pairs). The means for measuring may also function to measure the voltage across the total impedance of the coupling circuit (e.g., the whole circuit comprising the generator and all electrode pairs). Further, the means for measuring the voltage and/or current may be configured to measure the current within the at least one electrode (or the current supplied into a plurality of parallel electrodes). The means for measuring may also function to measure the current going through the total impedance of the coupling circuit. The voltage and/or current measurement may enable to track the applied (substantially) charge-balanced pulse, for example, during a medical treatment.

However, the voltage and/or current measurement may also be used for testing and/or calibration purposes that are not related to a medical process, as such. For example, the voltage and/or current measurement capabilities may be used for determining the impedance of the at least one electrode pair and/or the total impedance present in the coupling circuit (e.g., the whole circuit comprising the generator and all electrode pairs). Based on the determined impedance the total capacitance may be set (e.g. by the operator and/or automatically by the generator) which then may be set for the actual medical treatment. The (substantially) charge-balanced pulse used for determining the impedance may not have the same characteristics as a charge-balanced pulse that is applied during a medical treatment to avoid a medical reaction. To enable such a functionality, the electrical energy and/or power of the charge-balanced pulse applied may be set lower during an impedance measurement than during a medical treatment. For example, the amplitude of the internal pulse may be chosen lower for an impedance measurement than for a medical treatment. When the charge-balanced pulse may generally be used for an ablation treatment (e.g., an irreversible electroporation of tissue), the amplitude for the impedance measurement may be chosen such that an ablation threshold is not reached (e.g., within a margin of safety).

The means for determining the impedance may trigger the voltage and/or current measurements necessary for an impedance calculation. It may be conceivable that the impedance is determined based on a determined peak voltage, as well as the corresponding current. However, also more complex calculations may be implemented by the means for determining the impedance (e.g., calculating mean values, median values, applying a fitting algorithms, e.g. polynomial fitting algorithm, etc. of the voltage/current to determine an impedance value).

As described herein, the medical device may comprise one or more electrodes. For example, the medical device may comprise a catheter comprising the one or more electrodes. Notably, the generator may also be configured to determine the impedance (or an impedance value) of each electrode of the medical device. To enable such a functionality, one electrode of the medical device may be set as a reference electrode. For each remaining electrode the impedance is determined with respect to the reference electrode by applying a substantially charge-balanced pulse as a measurement pulse and determining the impedance as described herein. In particular, subtherapeutic charge balanced pulses may be used for determining the impedance.

In an example, the generator may not necessarily comprise the means for determining the impedance of the at least one electrode pair. It may also be conceivable that the generator may comprise only the means for measuring the voltage and/or current. For example, the means for measuring may still be triggered to perform measurements needed for determining an impedance. However, the actual calculation of the impedance may be performed by an external device that receives the impedance measurement values from the generator.

In an example, the internal pulse generator comprises a high-voltage source, wherein the generator is configured to form the internal pulse based at least in part on a high-voltage output of the high-voltage source, wherein the high-voltage source preferably can be set by the generator to provide a high-voltage amplitude of at least 1000 V, preferably at least 1500 V, more preferably at least 2000 V, most preferably at least 3000 V. The generator may thus enable to generate an internal pulse that comprises the high-voltage amplitude. The pulse-shaping output stage (or coupling circuitry) may then enable that a (substantially) charge-balanced pulse is generated having a corresponding high-voltage amplitude, as well. Notably, when the generator comprises the transformer configured as a step up transformer (as described herein) the voltage amplitude of the charge-balanced pulse may be even higher than the high-voltage amplitude provided by the high-voltage source. In an example, the high voltage amplitude provided by the generator may be in the range of 1000 V to 4000 V, 1500 V to 3500 V, 2000 V to 3000 V and/or 2500 V to 3500 V.

In an example, the internal pulse generator may comprise a high-voltage capacitor, wherein the high-voltage source is configured to charge the high-voltage capacitor. In that case, the generator may be configured to form the internal pulse based at least in part on the high-voltage amplitude of the charged high-voltage capacitor. For example, during a medical application the internal pulse generator may charge the high-voltage capacitor for a predetermined charging time such that the high-voltage capacitor may be considered to be essentially charged. One essential charge may enable to generate at least two internal pulses, preferably at least three internal pulses, more preferably at least four internal pulses, most preferably at least five internal pulses. Preferably, the capacitor system is fully charged between pulses, or, at least, between pulse trains (e.g. every heartbeat if the generator is sync-ed to the patient's cardiac cycle)

In an example, the internal pulse generator comprises a switching unit, wherein the switching unit is configured to switch the output of the high-voltage source to generate the internal pulse, wherein the switching unit preferably comprises an H-bridge circuit and/or a half H-bridge circuit. For example, the high-voltage source (and/or the high-voltage capacitor) may provide a steady output of a high-voltage amplitude. The switching unit may relay the high-voltage amplitude for a certain period of time to the pulse-shaping output stage in a first switch configuration. Subsequently, the switching unit may stop relaying the high-voltage amplitude to the pulse-shaping output stage in a second switch configuration. In an example, the second switch configuration may actively drive the input voltage at the pulse-shaping output stage to ground (e.g., to a potential of zero). Hence, an internal pulse with a defined internal pulse duration may be provided by the switching unit at the input of the pulse-shaping output stage.

The switching unit may also further comprise a power electronic circuitry or power electronic components such that the desired shape of the internal pulse may be created via the switching (e.g., a rectangular shape, a gaussian shape, a sinusoidal shape, a tooth shape, a sinc shape, etc.).

In an example, the internal pulse generator comprises a timing unit for controlling the switching unit preferably to set a timing parameter of the internal pulse and/or to set a number of internal pulses such that a train of internal pulses is applied to the pulse-shaping output stage. The timing parameter of the internal pulse may, for example, comprise a duration of the internal pulse and/or an interval between two internal pulses. The timing unit may further be configured to set a number of pulse trains wherein each train may comprise a specific number of internal pulses.

The internal pulse duration may be at least 0.5 μs, preferably at least 10 μs, more preferably at least 20 μs, most preferably at least 80 μs. Notably, the internal pulse duration set by the timing unit may be in the range of 0.5 μs to 200 μs, in the range of 1 μs to 100 μs, in the range of 1 μs to 80 μs, in the range of 1 μs to 50 μs, in the range of 1 μs to 30 μs.

The interval between two internal pulses may be at least 0.2 ms, preferably at least 0.4 ms, more preferably at least 5 ms, most preferably at least 10 ms. Notably, the interval between two internal pulses that may be set by the timing may be in the range of 0.1 ms to 20 ms, in the range of 0.2 ms to 15 ms, in the range of 0.3 ms to 12 ms, in the range of 0.4 ms to 10 ms, in the range of 0.5 ms to 10 ms.

The number of internal pulses per train may be at least 5, preferably at least 10, more preferably at least 100, e.g. at least 500. Notably, the number of internal pulses per train that may be set by the timing may be in the range of 5 to 600, in the range of 10 to 500, in the range of 20 to 500, in the range of 100 to 500, or in the range of 200 to 500.

In an example, the timing unit is configured to control the switching unit such that the internal pulse is applied based at least in part on a trigger of a medical signal. For example, the generator may be configured to receive a medical signal. The medical signal may comprise an electrocardiogram signal. In an example, the medical signal may be provided with a trigger signal which corresponds to a presence of a characteristic heart wave peak, a cardiac event and/or a cardiac cycle (e.g., an R wave peak, a QRS cycle, a P wave peak, a T wave peak, etc.). For example, the trigger signal may comprise a rectangular pulse signal wherein the rising edge of the trigger signal may correspond to a presence of the characteristic heart wave peak or cycle. For example, the internal pulse may be activated after a specific waiting time has passed after an R wave peak. The specific waiting time may be predetermined such that the internal pulses may be applied within a refractory period of the cardiac cycle.

In another example, the generator may be configured to determine the trigger in the received medical signal. Determining the trigger may be performed by the timing unit or any other suitable unit of the generator. For example, determining the trigger may comprise determining the characteristic heart wave peak, the cardiac event and/or the cardiac cycle via an according signal processing.

In an example, the generator is configured to apply a substantially charge-balanced pulse such that, if the at least one electrode pair comprises two electrodes of the medical device (e.g. in the form of a catheter system), the substantially charge-balanced pulse causes an irreversible electroporation (IRE) of a human tissue in the vicinity of (at least one of) the two electrodes. The human tissue may comprise a cardiac tissue, for example, of an atrium and/or a ventricle. However, the human tissue may also comprise a tissue of a vein and/or an artery (e.g., a pulmonary vein and/or a pulmonary artery). Notably, the generator may be adapted such that the ablation threshold for irreversible electroporation is fulfilled in the vicinity of at least one of the two electrodes. Notably, the generator may be used to drive a bipolar configuration of electrodes, as well as a unipolar configuration of electrodes of the medical device.

In an example, the generator may be configured to apply substantially charge-balanced pulses for a PFA treatment which may be based on irreversible electroporation. The generator may thus enable a controlled ablation of cardiac tissue, blood vessel tissue or any other tissue (e.g., nerve tissue, skin tissue, etc.) for a medical PFA treatment.

In an example, the generator, may comprise an interface unit for coupling to one or more external devices, such as a recording system that may for example record electrocardiographic obtained by the medical device. The interface unit may comprise an according output port for connecting to the one or more external devices. In an example, the interface unit may be configured to relay a signal of the at least one electrode pair to the output port (e.g. an electrocardiographic signal).

Notably, the herein described features of the generator and/or system according to other aspects of the invention may also be features and/or functionalities of the generator of the first aspect.

A second aspect relates to a catheter comprising: a connector for connecting electrode pairs of the catheter to a generator as described herein. For example, the connector may be configured such that the electrode pairs of the catheter match for a coupling with the pulse-shaping output stage. To that regard, the connector may be adapted to match with the interface relay board (as described herein), that may couple the electrode pairs to the pulse-shaping output stage of the generator in various ways.

A third aspect relates to a system comprising a generator as described herein (e.g. according to the first aspect) and a catheter as described herein (e.g. according to the second aspect).

A fourth aspect relates to a method for generating a substantially charge-balanced-pulse for application onto at least one electrode pair of a medical device, comprising: coupling the at least one electrode pair to an pulse-shaping output stage of a generator; applying a predetermined internal pulse with an internal pulse generator of the generator to generate a substantially charge-balanced pulse in the at least one electrode pair, wherein the method may be performed with a generator as described herein (e.g. according to the first aspect) and/or a system as described herein (e.g. according to the third aspect).

Notably, a further aspect relates to a computer program which may comprise instructions, that when executed by a computer, a generator of the first aspect, a catheter of the second aspect and/or a system of the third aspect, cause the computer, the generator, the catheter and/or the system to perform the method of the fourth aspect and/or a functional step associated with the method, as outlined herein. For example, the generator and/or the system may comprise means to execute the computer program instructions (e.g., a processing unit). The computer program may allow an autarkic, automated implementation of the aspects described herein. Consequently, technical intervention from medical staff may be minimized.

In an example, the computer, the generator, the catheter and/or the system may comprise one or more storage devices that may store one or more instructions that may be executed by the computer, the generator, the catheter and/or the system to perform a herein described method, functional step and/or operation of the generator, system and/or catheter.

A fifth aspect relates to a generator for a substantially charge-balanced pulse for application onto at least one electrode of a medical device, the generator comprising: means for determining an impedance of the at least one electrode by applying a substantially charge-balanced pulse. The impedance may comprise the impedance of a single electrode and/or an impedance value associated with the single electrode, for example that of the load associated with said electrode. However, the impedance may also comprise the impedance between a pair of electrodes or the total impedance of several electrode pairs. The impedance and/or the impedance value may be determined based on a current and/or voltage measurement as described herein. The aspects described above apply to both unipolar and bipolar ablation modalities.

In an example, the generator of the fifth aspect may be configured to send the determined impedance to a user interface, and/or display the determined impedance by the user interface. For example, the user interface may comprise a display (e.g., a monitor, a touchscreen, etc.) for displaying various information of the generator and/or the electrodes of the medical device.

In an example, also the generator of the fifth aspect may be configured to automatically set the total capacitance of the capacitor system comprised by the generator based at least in part on the determined impedance.

The generator according to the fifth aspect may also comprise features described herein with reference to other aspects.

In an example, a method may comprise (automatically) determining an impedance of at least one electrode and/or electrode pairs of a medical device as described herein. The method may further comprise (automatically) setting a (total) capacitance of an output capacitor system as described herein at least in part based on the determined impedance.

A sixth aspect relates to a system for coupling a substantially charge-balanced high voltage (electrical) pulse to a medical device, comprising: an output capacitor system which is configured for enabling at least two total capacitances. The system of the sixth aspect may comprise the pulse-shaping output stage as described herein. However, the system may also comprise the generator as described herein. In an example, the system of the sixth aspect may be implemented as a separate entity (e.g., as a separate board, as a separate device, etc.). In that case, the system of the sixth aspect may be for coupling to an input device wherein the input device may input the substantially charge-balanced high voltage pulse or another high voltage pulse to the system. The system may thus function as a (separate) intermediary device between, for example, the pulse generator and the medical device. Notably, the system may also transform the high voltage input pulse such that a substantially charge-balanced high voltage pulse is coupled to the medical device. As described herein, the output capacitor system of the system of sixth aspect may to that regard be coupled to an electrode pair of the medical device.

In an example, the system may be configured to set the total capacitance based at least in part on a number of electrodes of the medical device coupled to the system. The system may be configured for receiving a selection of electrodes via user interface and set the total capacitance based on the selection. The user interface may be configured to display only a selection of electrodes, in particular one or more selected sub-group(s) of all available electrodes. In another example, the system may be configured for an automatic detection of active electrodes to determine the number of coupled electrodes to the system.

In an example, the system may be configured to set the total capacitance based at least in part on a total impedance of one or more electrodes of the medical device coupled to the system. The system may be configured for receiving an impedance of the medical device and/or measure the impedance as described herein. For example, the system may comprise means for measuring the current and/or voltage of one or more electrode pairs of the medical device.

The generator according to the sixth aspect may also comprise features described herein with reference to other aspects.

A seventh aspect relates to a generator for substantially charge-balanced pulses for application onto at least one electrode of a medical device, wherein the generator is configured to apply the pulses in a manner triggered by an electro-cardiogram signal. The generator may be configured to receive the electro-cardiogram signal from an external source. The generator may thus comprise means for receiving the electro-cardiogram signal (e.g., an electronic receiving unit). The electro-cardiogram signal may comprise various signal channels which may comprise a trigger signal channel. In an example, the substantially charge-balanced pulses may be applied merely based on the trigger signal channel or the electro-cardiogram signal as such.

However, in another example the generator may be configured to receive the electro-cardiogram signal which may not necessarily comprise the trigger signal channel. Specifically, in that case the generator may comprise and/or implement an event detector that may determine cardiac events in the electro-cardiogram signal. In an example, the generator may comprise means for determining a characteristic heart wave event in the electro-cardiogram signal. In that example, the substantially charge-balanced pulses may be applied based on the detected events, e.g. as described herein. However, also a combination may be conceivable wherein the pulse application is based on the detected event by the generator and the electrocardiogram signal (e.g., the trigger signal channel).

In an example, the signal processing of the electro-cardiogram signal (and the corresponding control of the applied pulses) may be implemented by the timing unit of the generator. However, also any other computing entity of the generator may implement the signal processing of the electro-cardiogram signal (e.g., a synchronization unit, a central processing unit, a computer, a microprocessor, etc.).

In an example, the generator may comprise means for receiving a trigger instruction from a user interface, wherein the generator (or a computing entity of the generator) is configured to set a trigger signal based on the received trigger instruction and, e.g., the characteristic heart wave event (for example, the trigger signal may be set a certain amount of time after for example an R-wave peak). The pulses may then be applied by the generator according to the set trigger signal.

Notably, the generator of the seventh aspect may also comprise features or functionalities of the generator and/or system described with reference to other aspects of the invention. For example, the herein described functionalities with respect to the medical signal (in the first aspect) may be also applicable for the electro-cardiogram signal of the seventh aspect (and vice versa).

An eighth aspect relates to a generator for substantially charge-balanced high voltage pulses for application onto at least one electrode of a medical device, comprising a recording system for recording an electrical activity of the at least one electrode. The generator may further comprise an over-voltage protection element to protect the recording system from a high-voltage pulse applied to the medical device, preferably for an ablation of human tissue.

In an example, the recording of the electrical activity may be performed when no high voltage pulses are applied to the at least one electrode. The electrical activity may thus comprise the electrical activity sensed by the medical device. For example, the medical device may comprise a catheter wherein the electrodes of the catheter may also be configured for sensing, monitoring and/or mapping of electrophysiological activity (e.g., in the vicinity of the tissue contacted by the electrode). The recording system may, for example, also be used to record an electro-cardiogram signal (e.g., when the electrodes are positioned in a heart and/or in a vicinity of a heart). However, the recording system may be adapted to the voltage range and/or current range associated with sensing such that the high voltage pulse may damage the recording system. Hence, the over-voltage protection element may ensure that the recording system can reliably function over a prolonged period of time.

The over-voltage protection element may comprise one or more electrical elements to enable the protective function. For example, the over-voltage protection element may comprise switches and/or relays such that the electrode signal may be blocked during an application of pulses. The over-voltage protection element may also comprise one or more voltage suppressors (e.g., a transient voltage suppressor) to block any high-voltage signals that may be coupled to the recording system.

In another example, the recording system may be implemented by an external device which may be coupled to the generator. In that case, the generator may only comprise the over-voltage protection element such that a signal may be transmitted to the external recording device in a safe voltage range for recording purposes.

Notably, the herein described features of the generator and/or system of another aspect of the invention may also be features and/or functionalities of the generator of the eighth aspect.

It is noted that the method steps as described herein may include all aspects described herein, even if not expressly described as method steps but rather with reference to an apparatus (or device or system). Moreover, the generators (or systems or devices) as outlined herein may include means for implementing all aspects as outlined herein, even if these may rather be described in the context of method steps.

Whether described as method steps, computer program and/or means, the functions described herein may be implemented in hardware, software, firmware, and/or combinations thereof. If implemented in software/firmware, the functions may be stored on or transmitted as one or more instructions or code on a computer-readable medium. Computer-readable media include both computer storage media and communication media including any medium that facilitates transfer of a computer program from one place to another. A storage medium may be any available media that can be accessed by a general purpose or special purpose computer. By way of example, and not limitation, such computer-readable storage media can comprise RAM, ROM, EEPROM, FPGA, CD/DVD or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor. The control unit as described herein may also be implemented in hardware, software, firmware, and/or combinations thereof, for example, by means of one or more general-purpose or special-purpose computers, and/or a general-purpose or special-purpose processors.

### DESCRIPTION OF THE DRAWINGS

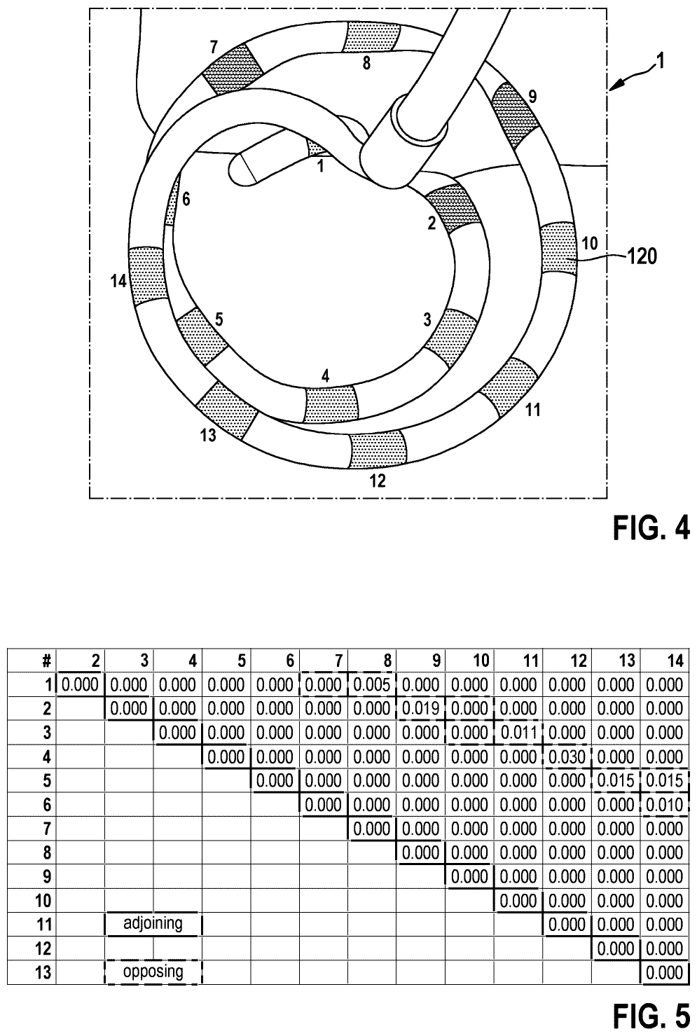

Turning now to the drawings, FIG. 1 and FIG. 4 illustrate a distal portion of an ablation catheter 1 in accordance with a first embodiment. The ablation catheter may be used for PFA, when used with the PFA generator and accessories, and is indicated for use in cardiac electrophysiological mapping (stimulation and recording) and in high-voltage, pulsed-field cardiac ablation. Peak voltages are, for example, without limitation, +/−1 kV to 3 kV with a pulse width of up to 30 μs. Higher peak voltages (e.g. up to 10 kV) may be used provided the pulse duration is correspondingly shorter (e.g. 0.5 μs). The catheter 1 has an elongated circular catheter shaft 10, which may connect with a handle comprising a steering mechanism at a proximal end (not illustrated). As a result, the catheter may control deflections of the depicted distal section carrying the ablation electrodes.

At the illustrated distal end of the catheter shaft 10 an ablation portion 12 is arranged, which comprises a plurality of loop sections 121, 122. The concept of loop sections includes embodiments that use continuous loops or spirals configurations. The catheter shaft may have an effective length of approximately 115 cm from the distal tip of the ablation portion 12. Each of a first loop section 121 and a neighboring second loop section 122 exhibits ablation electrodes 120 (altogether, for example, 14 electrodes), which are configured for delivering energy to tissue. Although two loops are illustrated in FIG. 1, more can be used. It is preferred that at least a partial third loop is used in order to provide sufficient overlap among resulting ablation zones. Said overlap would increase chances of achieving a conduction block moat without drops in lesion continuity, contiguity or transmurality. The distal section comprises at least 45° of overlap of a 3.sup.rd loop section with the previous two sections. In particular, the ablation catheter 1 may be configured for delivering an electrical high voltage PFA signal to tissue via the ablation electrodes 120. For example, the ablation electrodes 120 may consist of or comprise gold and/or a platinum/iridium alloy. Alternatively, electrodes 120 from different loop sections may be positioned so that electrodes of same polarity are aligned. However, dependent on the form of the patient's tissue and the position of the ablation portion 12, electrodes of opposite polarities may collide when the spiral catheter is compressed thereby causing arcing and/or the contact of the electrodes with the patient's tissue may not be uniform. In the exemplary embodiment illustrated in FIG. 1, the ablation electrodes 120 of the second loop section 122 are arranged partly in a staggered manner with respect to the ablation electrodes 120 of the first loop section 121.

In order to address measurement values to the different electrodes 120, the electrodes are consecutively numbered as shown in FIG. 4 (see numbers at the electrodes). The most distal electrode has the number 1, whereas the most proximal electrode is denoted with number 14. Different numbering is possible, as well.

The loop sections 121, 122 may further exhibit a plurality of mapping electrodes, which are configured for receiving electrical signals from tissue.

Together, the loop sections 121, 122 form a three-dimensional spiral, which form a corkscrew-similar form. Alternatively, they may form a plunger-like configuration or any other suitable 3-dimensional configuration (not shown).

The loop sections 121, 122 may comprise a shape memory material, for example, in the form of an inner structural support wire (not illustrated), for example a Nitinol wire as described above. In particular, the loop sections 121, 122 may have super-elastic properties.

The ablation portion 12 may be constrained into an essentially elongate shape for the purpose of delivery to a target region in the human body by means of a (fixed or steerable) delivery sheath 15, which may also be referred to as an introducer sheath. At the target position, upon exiting a distal end of the delivery sheath 15, the ablation portion 12 may then recoil to its original (biased) shape.

The length of each electrode 120 along the respective loop section 121, 122 is, for example, 4 mm. In general, the electrode length is in the range 1-10 mm, preferably 3-5 mm. The catheter shaft 10 size may be compatible with an 8.5 F ID sheath and may consist of radiopaque extrudable polymer and, if applicable, a polymer-reinforcing braid. In general, the size of the catheter shaft 10 may be compatible with a 7 F to 14 F ID sheath. The width between neighboring electrodes along the respective loop section may be chosen between 1 mm and 10 mm, preferably 3-6 mm, in order to provide a contiguous ablated area at the patient's tissue.

FIG. 2 schematically and exemplarily illustrates a delivery path for an ablation catheter 1 leading to a pulmonary vein ostium (PVO) of a human heart. For orientation, the inferior vena cava (IVC), the right atrium (RA), the right ventricle (RV), the left atrium (LA), the left ventricle (LV), as well as pulmonary veins (PV), each with a PVO, are shown. The large black arrows indicate a delivery path passing through the IVC, the RA, transeptally through the septal wall (SW), and into the LA. Finally, using appropriate deflection means, catheter 1 is steered to PVO regions. There, the corkscrew type ablation catheter may be used for ablation in the area of the atrial end of the pulmonary vein close to PVO. The form of the ablation portion 12 is configured such that it fits to the dimensions of the targeted PVO. Alternatively, corkscrew-type catheters may be used to ablate at the SVC or at Appendages, such as the left or right atrial appendages (LAA or RAA).

Reliable full ablation along a whole circumference is achieved with the first embodiment of the ablation catheter shown in FIGS. 1 and 4 at their respective position within the heart or the vein to which the form is adapted. A small compression of the ablation portion 12 of the respective catheter 1 may be possible during ablation into the direction of the longitudinal axis of the spiral.

The ablation procedure using one of the ablation catheters 1 may start after the ablation portion 12 is in the correct position relative to the targeted tissue, for example at a PVO. The assessment of the position and/or configuration of the ablation electrodes 120 is provided prior and/or between two ablation steps (if applicable) and is explained in more detail below. The ablation electrodes 120 will provide pulsed electric RF field in a unipolar or bipolar arrangement. Peak voltages are, for example, without limitation, +/−1 kV to 3 kV with a pulse width of up to 30 μs. Higher peak voltages (e.g. up to 10 kV) may be used provided the pulse duration is correspondingly shorter (e.g. 0.5 μs). The pulse width may be 12 μs (between 0.5-30 μs) forming a pulse train comprising up to 500 pulses/train.

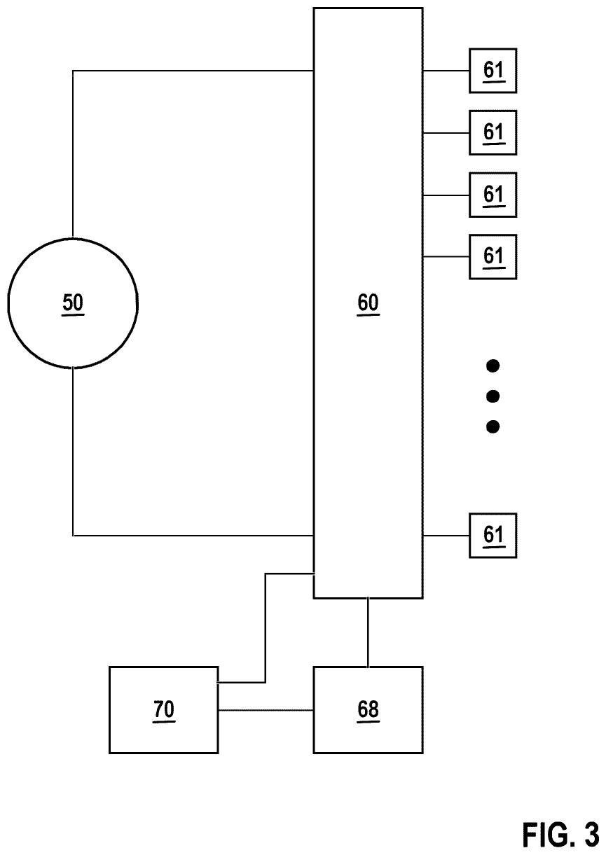

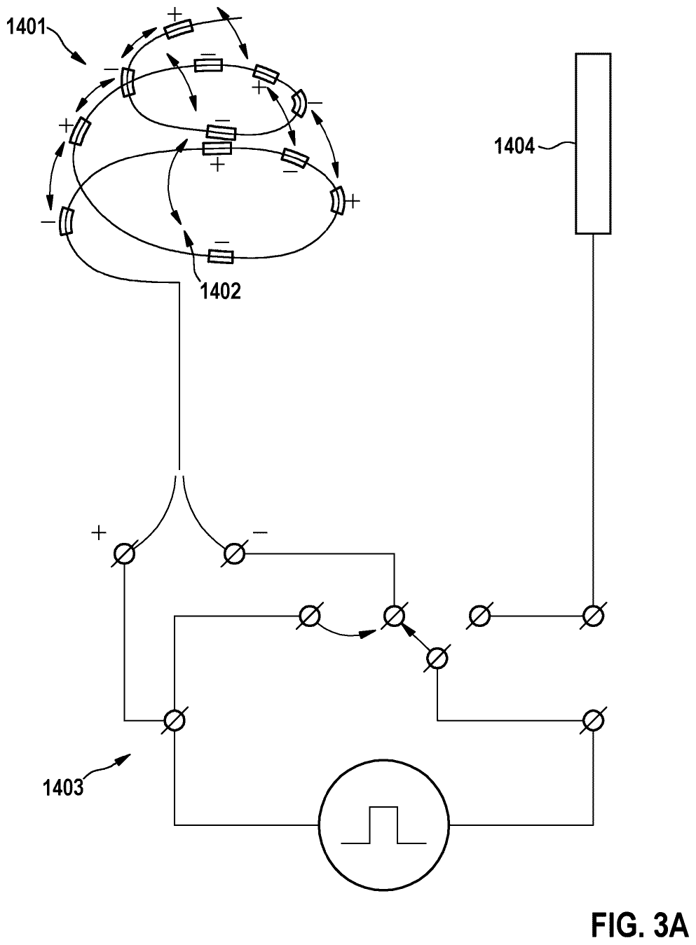

The electric field generation (in particular voltage, current and impedance) is monitored by an electronic control unit (ECU) 70 which is connected to the leads 61 of the electrodes 120 and produced by a waveform generator 50 (see FIG. 3). FIG. 3A also shows connectivity that can be used to generate unipolar or bipolar electric fields. ECUs in FIGS. 3 and 3A may control application of PFA fields. FIG. 3A illustrates a catheter 1401 (such was the one with reference number 1 from FIGS. 1 and 4) with its electrodes driven by ECU 1403. ECU 1403 can be controlled to deliver field vectors 1402 that cover the tissue zone in between catheter 1401 spiral arms/loops. By doing so, the AR index may be determined. In order to provide quasi-unipolar measurements, the PFA generator may be connected to one of the electrodes as the reference electrode instead of to the grounding pad 1404.

In order to assess the positions and/or configuration of the electrodes 120 with regard to each other and the targeted tissue, the ablation catheter further comprises a measurement unit 68 which is connected to the ECU 70 and a switch unit 60 with the waveform generator 50. The measurement unit 68 is configured to measure peak current and peak voltage as well as impedance at the respective electrode lead 61 and transmit these data to the ECU for further analysis. Further, the measurement unit 68 provides the electrodes 120 at the respective lead(s) 61 with pre-defined measurement signals (current or voltage pulses) via the waveform generator 50 in order to measure the above-mentioned parameter.

In the bipolar arrangement neighboring (adjoining) electrodes 120 may be paired along the loop sections 121, 122, across two neighboring loop sections 121 and 122 or any other pre-defined pair combination, in particular for impedance determination for AR value and/or CU value. Further, the electrodes 120 may be used in a unipolar arrangement. In this case, a ground pad 1404 may be provided at the surface of the patient's body. Alternatively, one of the non-adjacent electrodes 120 may be used as reference electrode thereby forming a quasi-unipolar arrangement.

In order to switch between different bipolar arrangements or between unipolar and bipolar arrangement, the ablation catheter 1 may comprise a switch unit 60 connected to and controlled by the ECU 70. The switch unit 60 provides the respective phase of the pulsed electric field provided by the waveform generator 50 to the predefined electrode lead 61 and thereby to the predefined electrode 120 wherein each electrode lead 61 is electrically connected to one particular electrode 120 at the ablation portion 12. The switch unit 60 comprises a switch matrix and may realize any configuration of phase distribution, for example, such that two neighboring electrodes along the loop sections, across the loop sections and any other electrodes are paired. The switching signal and configuration information is provided by the ECU 70. ECU 70 further may provide data processing of electrical or biopotential data or impedance data acquired the electrodes of ablation catheter 1. As indicated above mapping electrodes located in the ablation portions 12 may comprise mapping electrodes for determining the electrical potential of the surrounding tissue in order to observe the ablation progress at pre-defined time points during ablation procedure. Alternatively, the ablation electrodes 120 may be switched into the mapping mode and back into the ablation mode.

As indicated in the general description, prior ablation treatment and/or between ablation treatment steps the AR value and CU value are determined in order to assess the positions of the electrodes 120 and/or their configuration with regard to each other and/or with regard to the tissue under treatment.

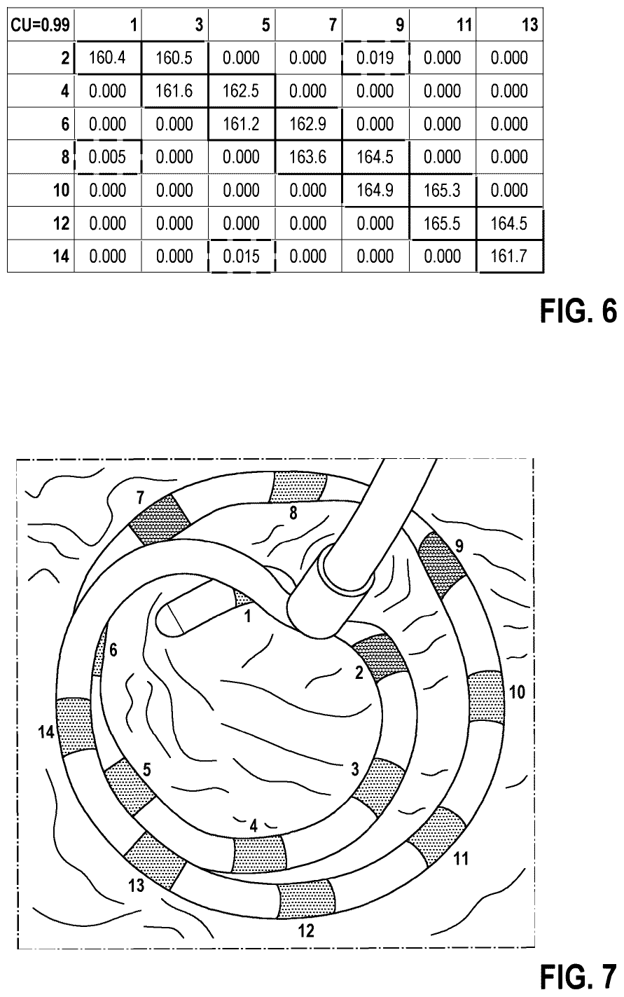

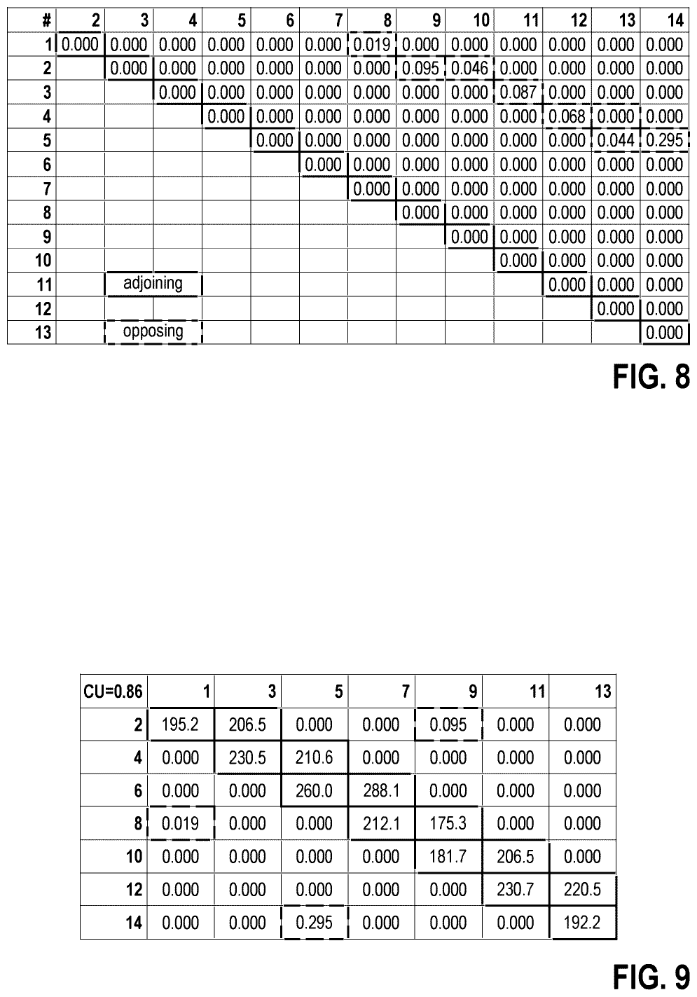

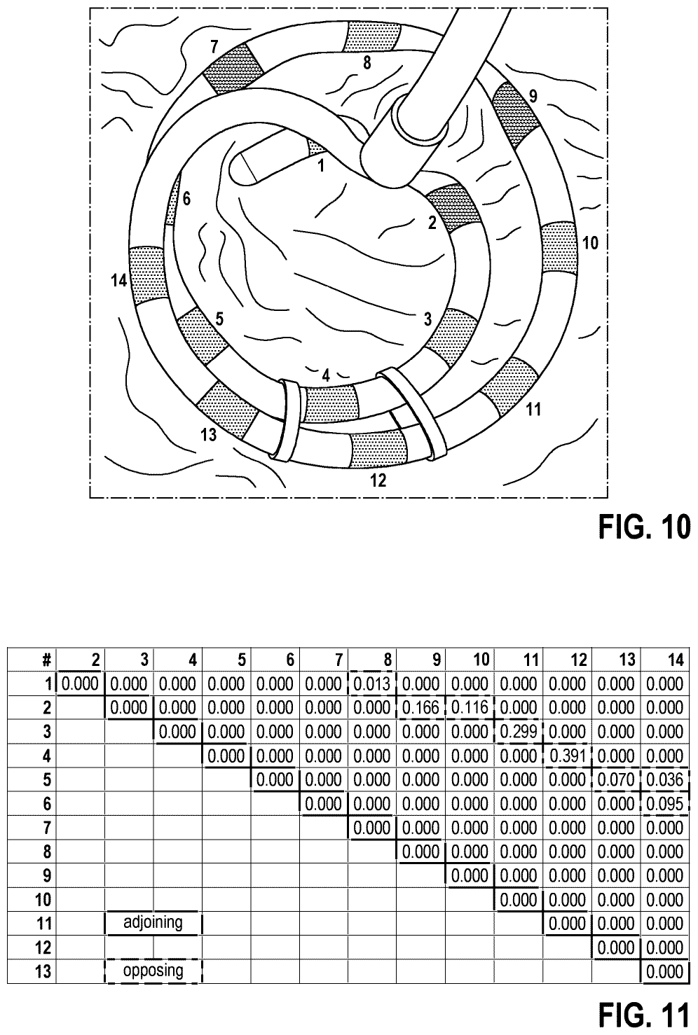

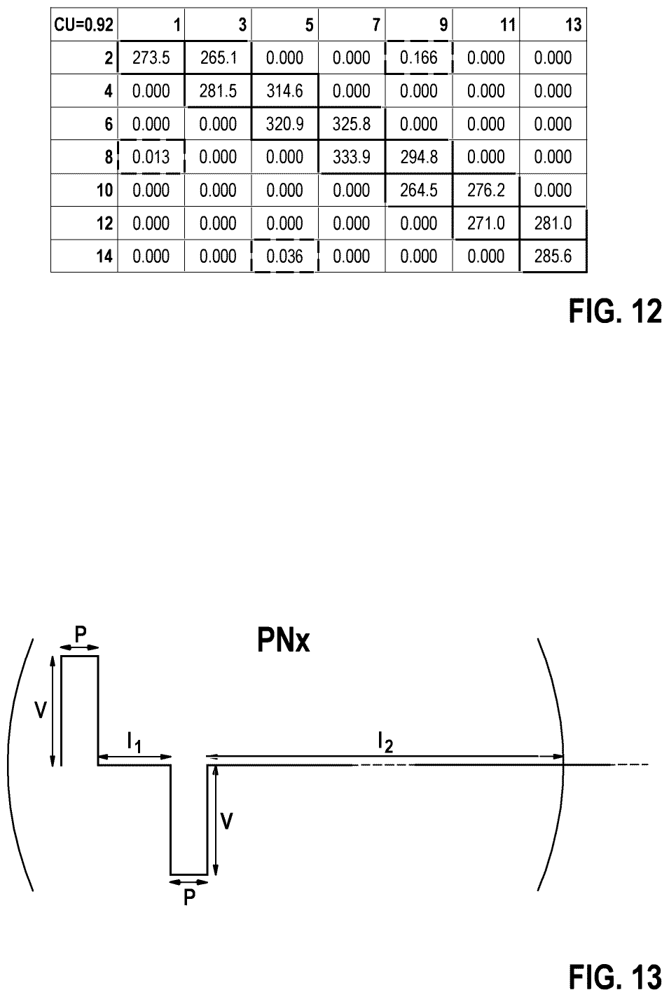

In the first example, the ablation catheter of FIGS. 1 and 4 is measured with regard to the impedance of all pairs of the 14 electrodes in saline (for comparison), a first position axially pressed to a chicken heart tissue (see FIG. 7) and in a second position axially pressed to a chicken heart tissue wherein black rubber bands keep the electrodes 4 and 12 close to each other (see FIG. 10). The matrices of FIGS. 5 and 6 belong to the saline configuration, the matrices of FIGS. 8 and 9 to the position shown in FIG. 7 and the matrices of FIGS. 11 and 12 to the position shown in FIG. 10.

For example, AC voltage signals with a frequency of 500 kHz with a peak voltage (amplitude) of 1 V are chosen. The matrices of FIGS. 5, 8 and 11 show the AR index calculated from the bipolar impedance measurement values Z.sub.x,y of the electrode pair x,y. The number of the electrodes of the particular electrode pair can be found in the respective header line and the first row. The value at the row-line-intersection contains the AR index of the respective electrode pair x,y determined from the impedance measurement values for 500 kHz. The AR index is calculated using the formula

$$AR x , y = 1 - Z x , y min ( Z x - 1 , x , Z x , x + 1 , Z y - 1 , y , Z y , y + 1 ) .$$

All AR index values are zero or close to zero for the saline configuration. No risk or arcing exists since all electrodes have a sufficient distance to each other.

In contrast, with regard to the ablation portion position of FIG. 7 it is apparent that the AR index of the electrode pair 5, 14 is considerable higher than the other AR indexes. In FIG. 7 it appears, that these electrodes are the only ones which are close to each other—there is an arcing risk with regard to these electrodes and repositioning is needed.

The matrix of FIG. 11 contains the AR index values calculated in a similar way for the configuration of FIG. 10 and a frequency of 500 kHz. It is apparent that in particular the electrode pairs 3, 11 and 4, 12 show considerable higher AR index values than any other AR index value of this matrix. For these pairs a risk for arcing exists, if the electrodes of these pairs would be at different polarities.

In another representation shown in FIGS. 6, 9 and 12 the calculated AR indexes of the respective electrode pairs (electrode numbers are shown in the header line and in the first row, formula see above) are provided for all electrode pairs but the adjoining electrode pairs (marked in the diagonal) for the respective ablation portion position. In the diagonal line the impedances of the adjoining electrode pairs are provided. In the matrix of FIG. 9 the AR index of the electrode pair 5 and 14 is highlighted since it indicates a high arcing risk (AR index>0.25). With regard to the third position (FIG. 10), in particular, the electrode pair 2, 9 has a higher arcing risk. Just for clarification, in this position the AR index values for the electrode pairs 4, 12 and 5, 13 are neglected since these electrodes share the same polarity and therefore no risk for arcing exists.

Further, the diagrams of FIGS. 6, 9 and 12 contain the CU value for the respective position in the upper left corner calculated from the following formula (see explanation above) and the measured bipolar impedances of the adjoining electrodes

$$CU = 1 - σ ( \{ Z n , n + 1 \} ) μ ( \{ Z n , n + 1 \} ) .$$

It appears from the matrices in FIGS. 6, 9 and 12 that the contact uniformity of the position shown in FIG. 7 is better than of the position shown in FIG. 10 as the CU value is greater (0.92>0.86). The contact uniformity is best in the saline position (0.99)—if all electrodes without contact, i.e. all electrodes are floating in saline.

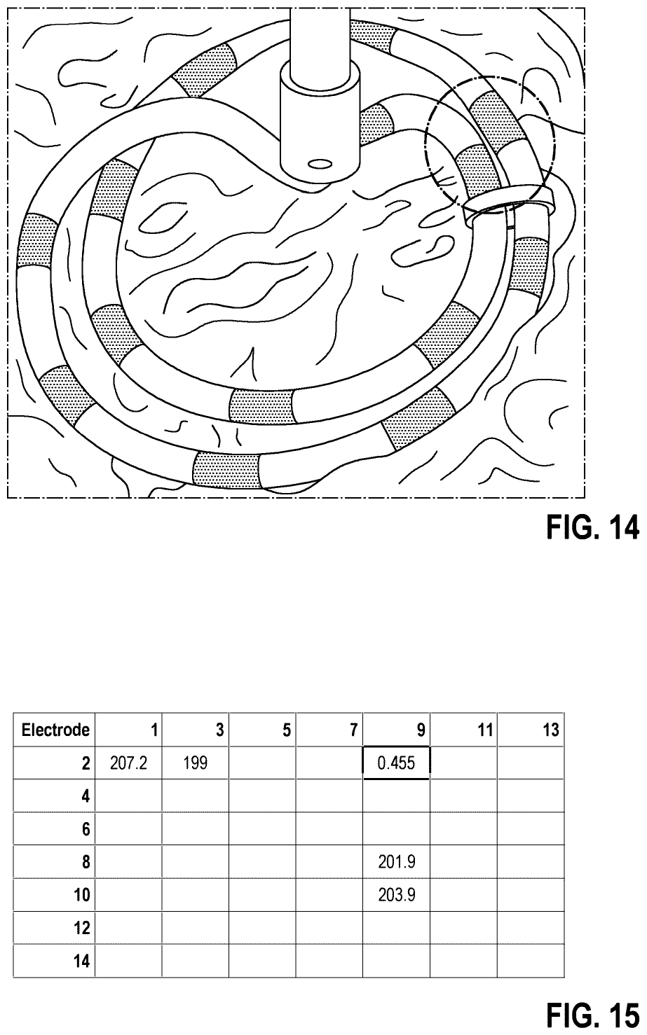

Further examples of ablation catheter positions pressed to a chicken heart are shown in the following FIGS. 14 to 19, wherein a profile shown in FIG. 13 is used as PFA protocol, wherein V=2.5 kV, P=3 μs, I.sub.1=25 μs, and I.sub.2=2 ms. Further, a pulse number PN=20 were chosen intentionally to provoke arcing.

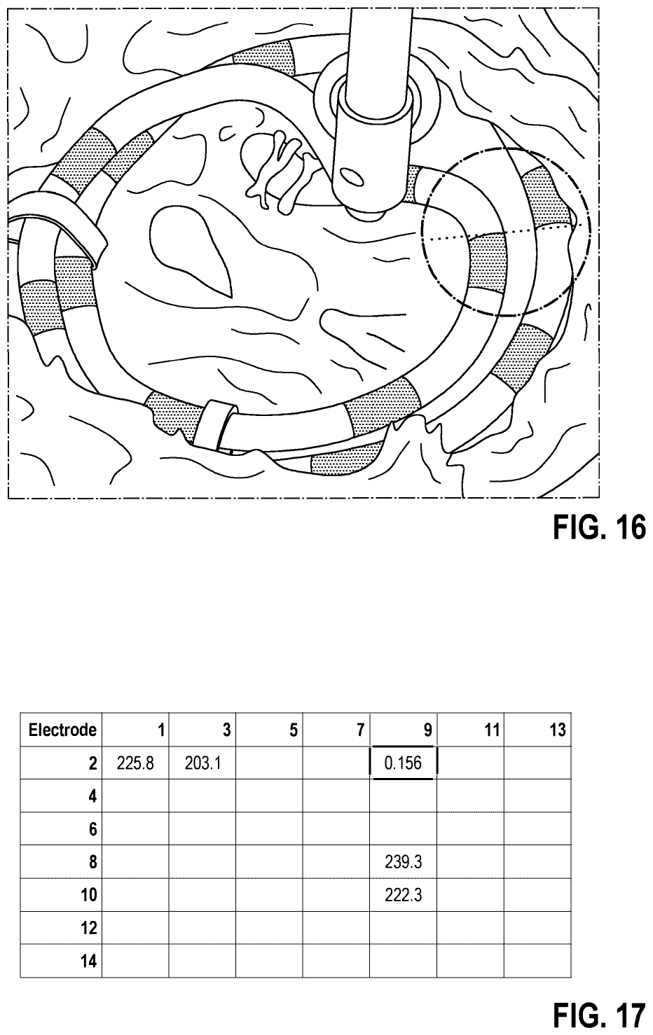

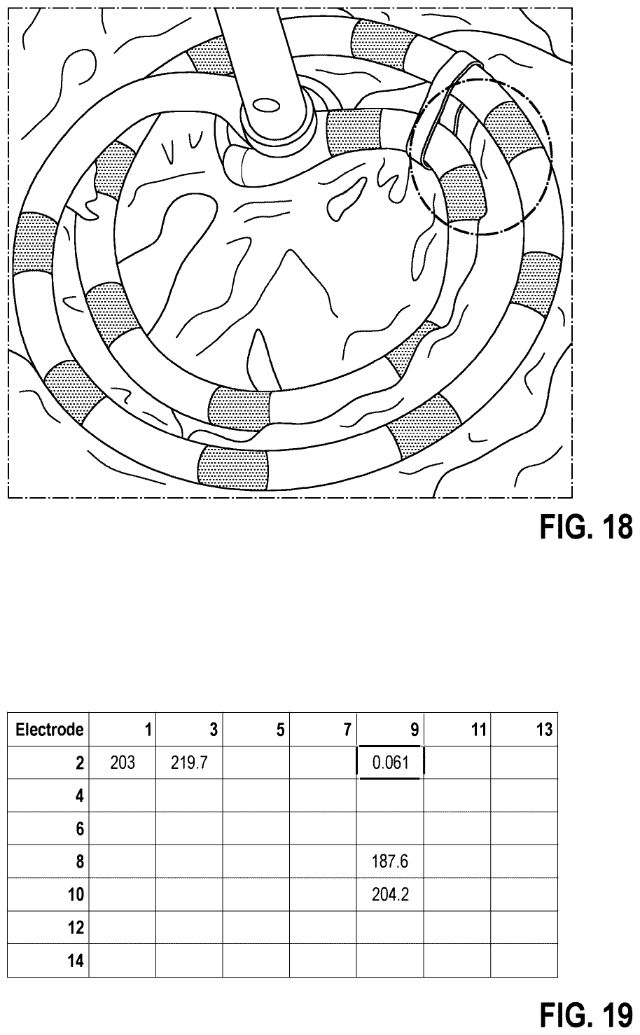

FIG. 14 shows a position of the ablation portion of the ablation catheter of FIGS. 1 and 4 in which the electrodes 2, 9 are in close proximity (see encircled area). Accordingly, the AR index of these electrodes is 0.455 indicating the high arcing risk (see matrix shown in FIG. 15). The arcing threshold was determined as 0.9 kV confirming the calculated AR index. FIG. 16 shows a position of the ablation portion of the ablation catheter of FIGS. 1 and 4 where electrodes 2, 9 do not overlap (see marked area, so-called edge-edge position). Accordingly, the AR index shown in FIG. 17 is lower than the one of FIG. 15. The lowest AR index may be found for the position of these electrodes 2,9 shown in FIG. 18 in which these electrodes are sufficiently far away thereby having a low arcing risk (see marked area). Accordingly, the AR index of this electrode pair 2, 9 is close to zero (see FIG. 19).

In another example, the CU value for two positions of the ablation catheter of FIGS. 1 and 4 is determined, in particular the CU value determined from bipolar impedance measurements of adjoining electrodes using formula (n=1 . . . 13)

$$CU = 1 - σ ( \{ Z n , n + 1 \} ) μ ( \{ Z n , n + 1 \} )$$

is compared with the CU value determined from quasi-unipolar impedance measurement values. For determination of the CU value for the quasi-unipolar impedance measurement values Z.sub.n in the above formula the parameter Z.sub.n,n+1 is replaced by Z.sub.n for the standard deviation and the mean value. In this case n=1 . . . 14. The quasi-unipolar impedance one electrode (e.g. electrode 1) is measured against all electrodes of opposing polarity (e.g. against all even electrodes, and electrode 2 against all odd electrodes).

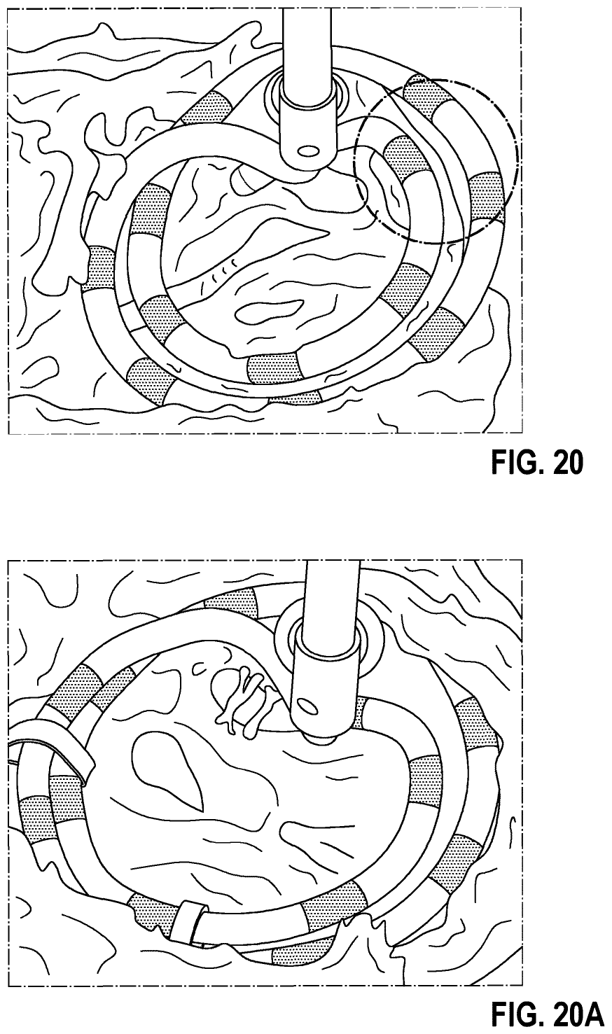

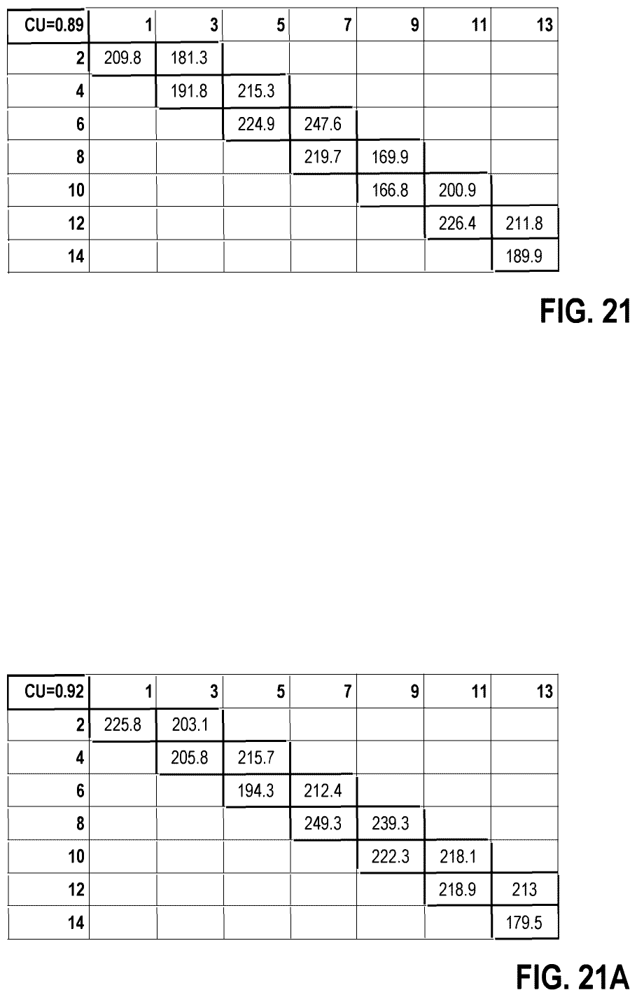

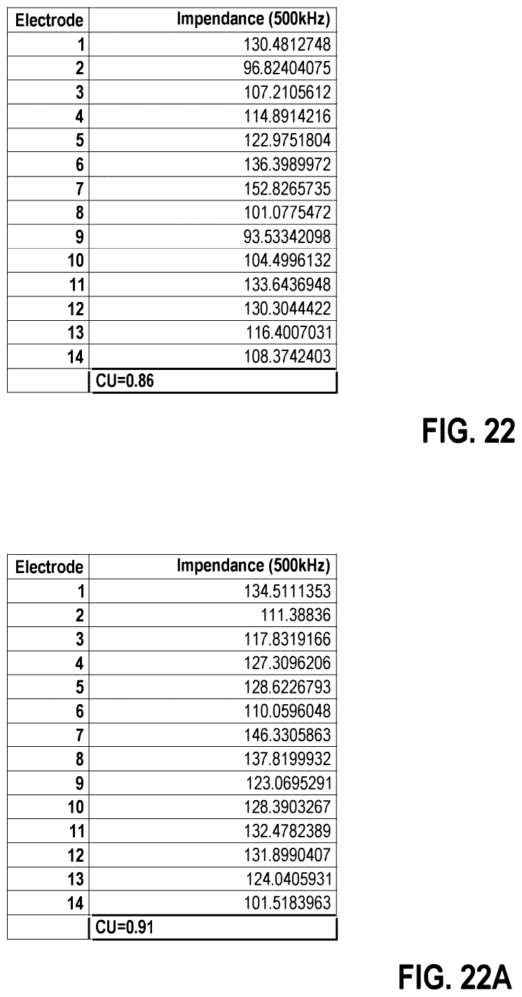

FIG. 20 shows a position in which three electrodes (2, 8, 9) are floating in saline while the others are in contact with the heart tissue. The CU value (bipolar, see FIG. 21) is 0.89 and the CU value (quasi-unipolar) is determined as 0.86 (see FIG. 22) which is comparably low thereby indicating bad contact uniformity. In contrast the position shown in FIG. 20A has all electrodes in contact with the chicken heart's tissue. Accordingly, CU value (bipolar, see FIG. 21A) is 0.92 and the CU value (quasi-unipolar) is determined as 0.91 (see FIG. 22A).

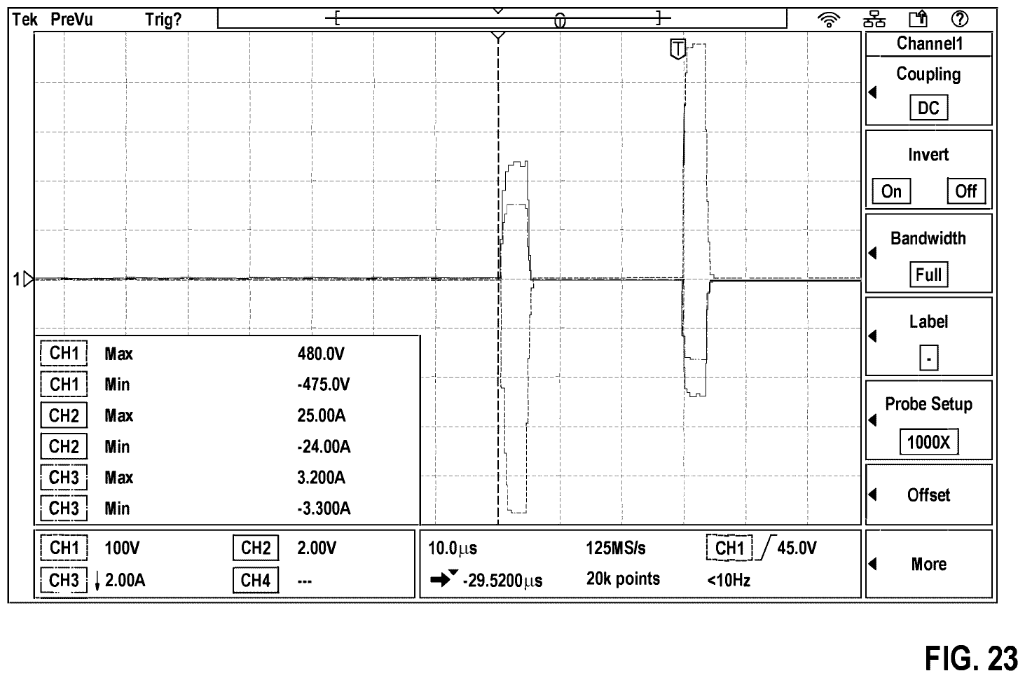

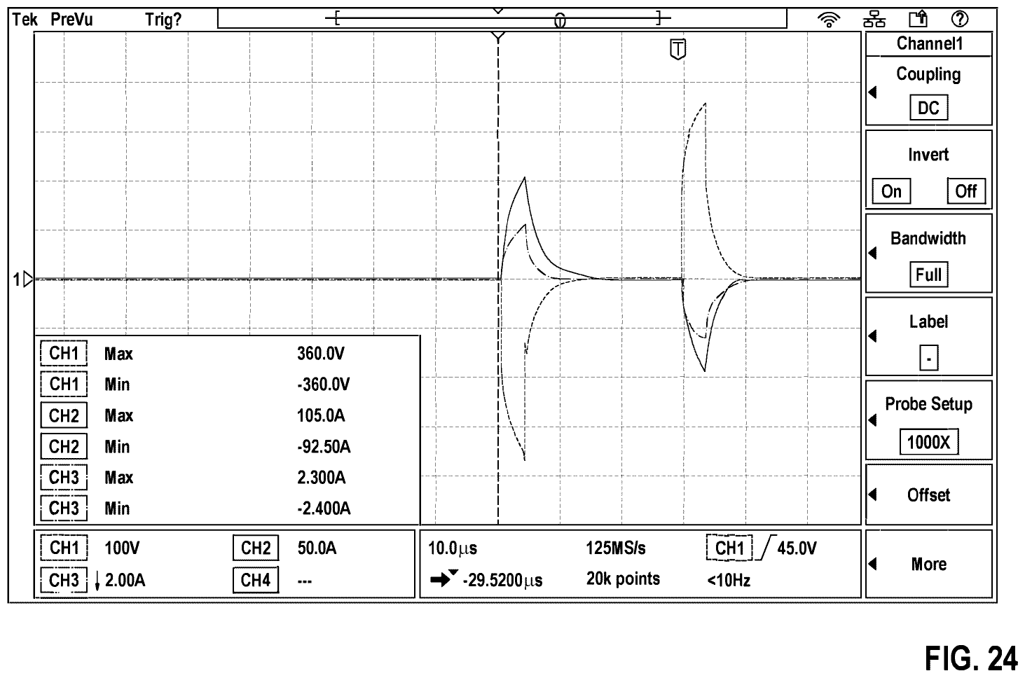

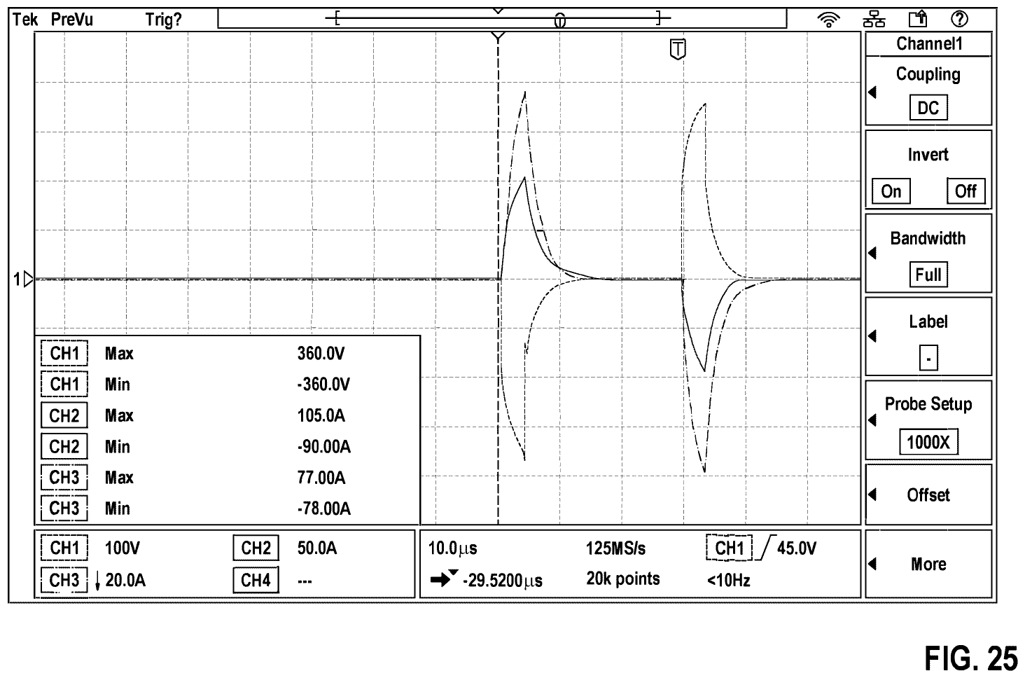

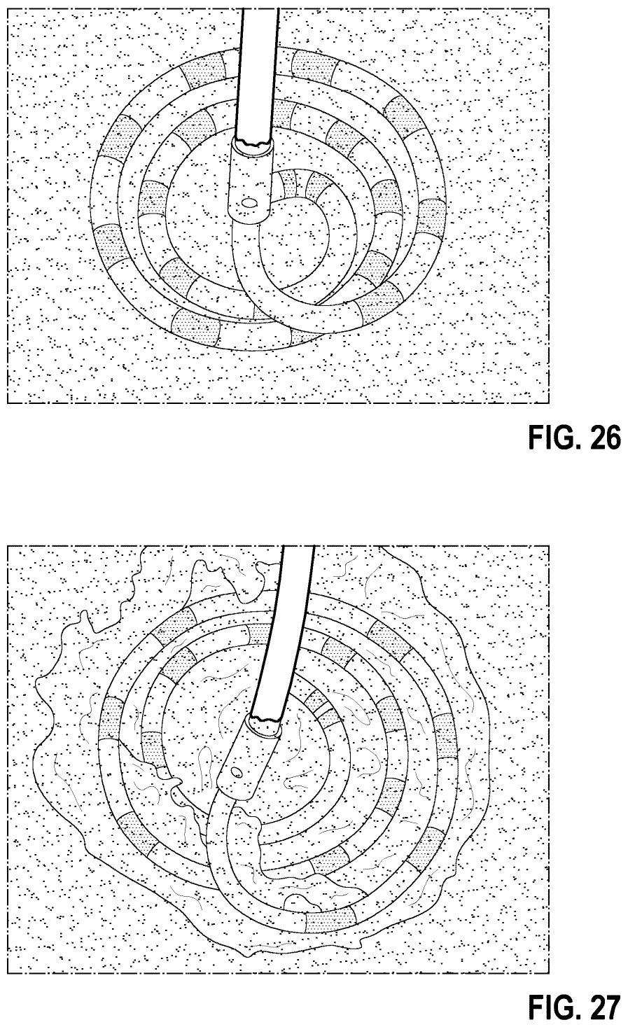

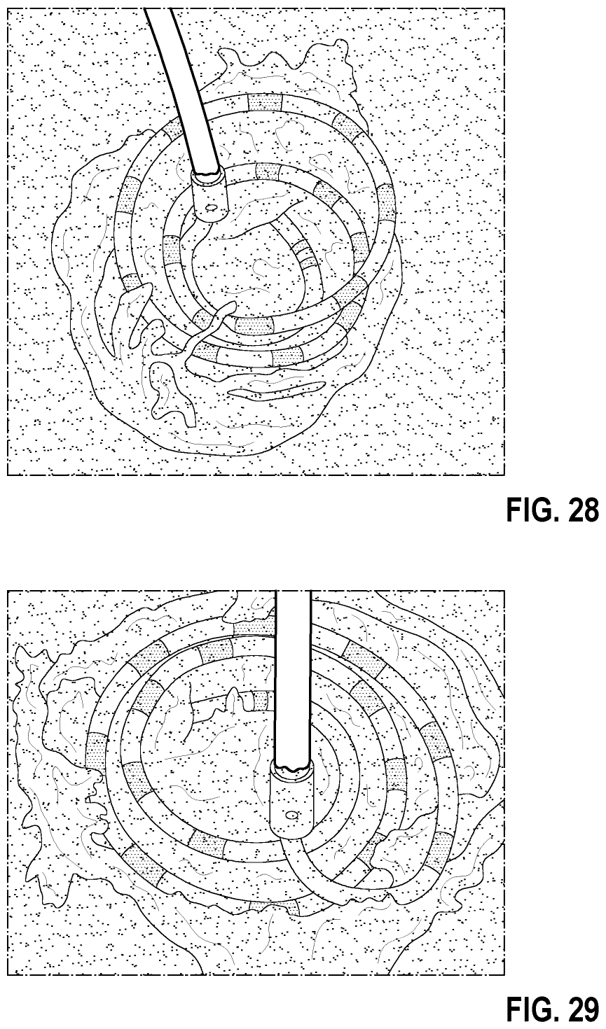

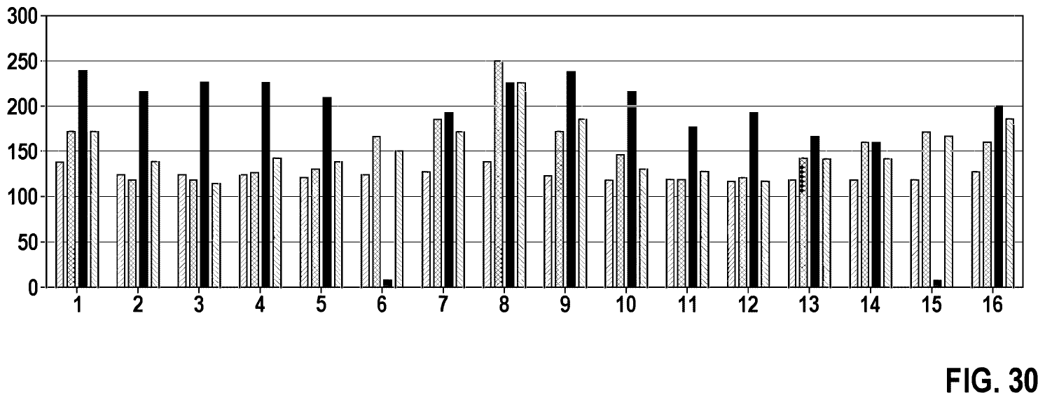

FIGS. 23 and 24 show the current measurements using a single pulse for each of the electrodes in order to determine CU, namely a rectangular pulse. FIG. 23 represents a rectangular current waveform as response to the rectangular voltage pulse. The tooth shaped waveform shown in FIG. 24 represents the measured current in the case of a short circuit. Even in this case a current measurement and thereby impedance measurement is possible. Current measurements (all even electrodes, 16 single electrodes) have been performed with a current transformer (Magnelab CT-C0.5) while a 500 V rectangular biphasic pulse (4 μs pulse length, 25 μs interphase delay) was applied. The impedances determined from the peak current measurement values are displayed as bars for each electrode (electrode number at x-axis) and impedance (in Ω at y-axis). The first (dark blue) bars refer to the position shown in FIG. 26 (ablation portion in saline), the second (orange) bars refer to the position shown in FIG. 27, the third (grey) bars refer to the position shown in FIG. 28, and the fourth (yellow) bars refer to the position shown in FIG. 29.

The impedance values shown for the saline configuration are low because of the higher conductivity of saline (^{˜}0.7 S/m, which is matched to human blood in this experiment) compared to the chicken heart tissue. For the position shown in FIG. 27 the electrodes 2 to 5 and 11 to 13 have lesser contact, whereas the other electrodes have better contact. Regarding the position shown in FIG. 28 the electrodes 6 and 15 are short circuited and the position of the ablation portion needs to be corrected (impedance close to zero). The position shown in FIG. 29 provides impedance values similar to the position of FIG. 27.

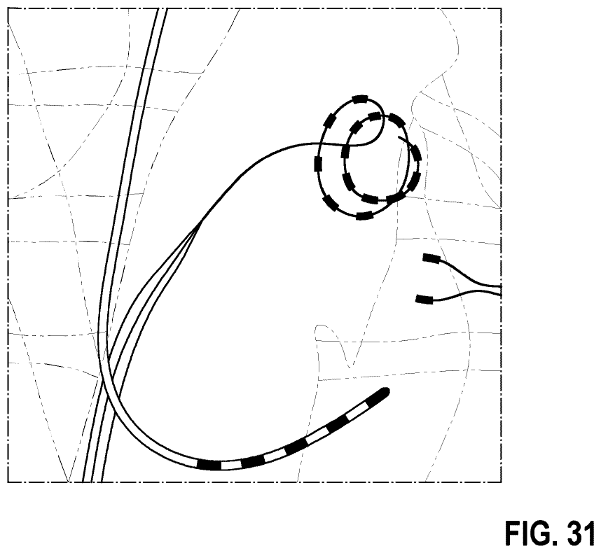

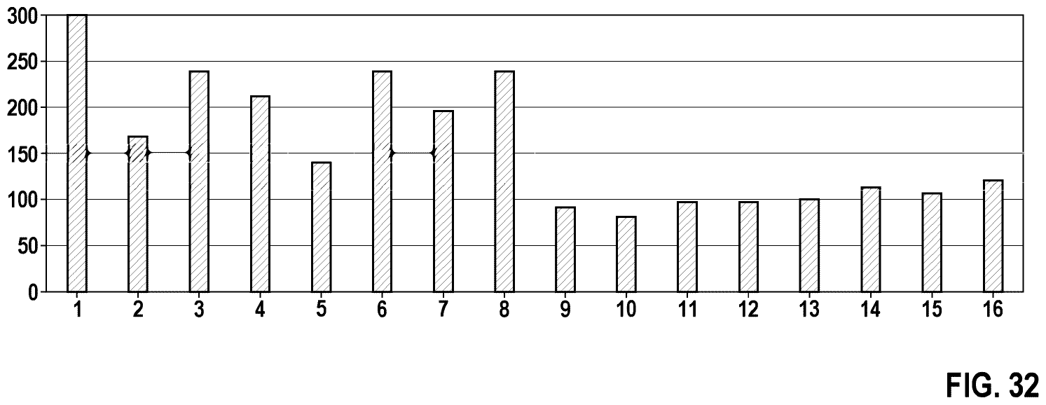

FIG. 31 shows an animal setup. For this test a corkscrew-type catheter (25 mm outer diameter) with 16 electrodes with a spacing of 6 mm (first group of 8 electrodes) and 3 mm (second group of 8 electrodes) was positioned at the right ventricular outflow tract of an 80 kg pig. Rectangular pulses with an amplitude of 500 V were used. For calculating impedance, the maximal values of voltage and current were used. FIG. 32 shows the impedance values (in (2) determined from current measurements as bars in relation to the respective electrode (see x-axis). It can be shown that the electrodes 9 to 16 have a quite good contact uniformity, whereas with regard to the electrodes 1 to 8 the contact uniformity can be considered mediocre. However, the impedance values of the first group of electrodes 1 to 8 is higher as the electrodes are at a greater distance compared to the second group of electrodes. Accordingly, if one calculates the IU value for the electrodes, the two groups of electrodes should be differentiated. If one applies the above formula for IU and IU*, one derives IU=0.84 and IU*=0.58. It can be seen that IU* appears to be too low as it does not take the two groups of electrodes into account.

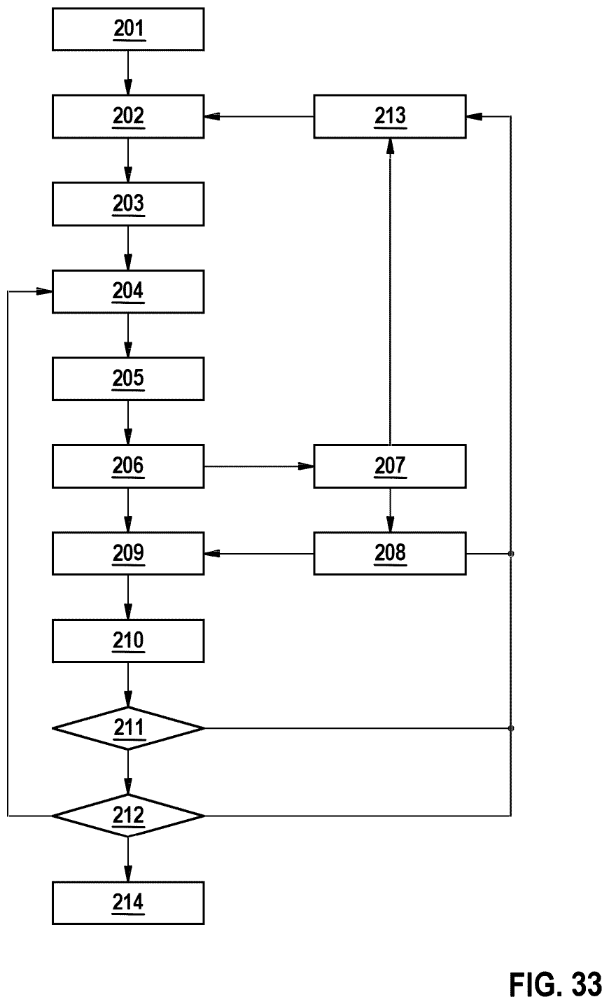

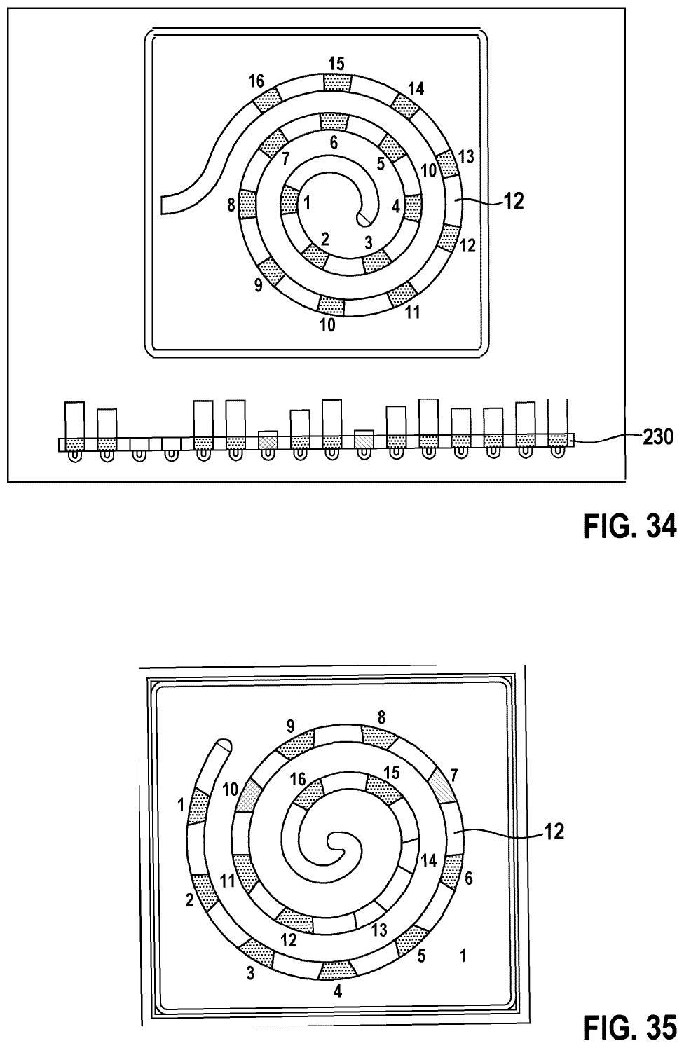

In the following the usage of an inventive catheter as described with regard to FIGS. 1 and 4 is explained in detail referring to the flowchart of FIG. 33. In the first step 201, the catheter 1 is manipulated to targeted PV antrum in the usual way. During advancement of the catheter the ablation portion 12 is covered by the delivery sheath 15 until the distal end of the catheter reaches the targeted region. In the next step 202, the catheter provides quality EGMs to confirm placement near PV and to assess pre-PFA amplitudes and/or an electro-anatomical mapping system displays the 3-dimensional shape and location of the catheter 1. Then, in the next step 203, and after release of the ablation portion 12 from the delivery sheath 15 by retracting the delivery sheath into proximal direction, the AC index and/or CU value measurement is started by short pressing a food pedal of the catheter 1. Then, in step 204, accurate current or impedance measurements between electrodes 120 of the catheter are provided as explained above in detail by the measurement unit 68, the waveform generator 50 and the ECU 70. In one embodiment, the measurement may be provided to all electrodes 120 of the ablation portion 12 or, alternatively, electrodes at positions at risk are measured. Afterwards, the current or impedance measurement values are processed by the ECU 70 and the impedance values for all ablation electrodes, AR indexes of electrode pairs, IU value and/or the CU value for all ablation electrodes of the ablation portion 12 are determined in the following step 205. In step 206, the GUI connected with the ECU 70 colors catheter electrodes or a respective bar diagram at risk of arcing in easy-to-see colors as shown in FIGS. 34 and 35. FIG. 34 depicts the ablation portion 12 with 16 numbered electrodes 120 and a respective bar diagram 230, wherein the height of a bar shown with reference to the electrode number represents the impedance value. The bar diagram shows a low impedance for electrodes number 7 and 10. Electrodes 13 and 14 are mapping electrodes and therefore not measured. FIG. 15 indicates the calculated impedance values directly at the electrode location of electrodes 7 and 10 at the ablation portion 12 with different colors, wherein each color represents the deviation from the target impedance value. The red color of electrode number 10 visualizes a greater deviation from the target impedance value than the yellow color of electrode number 7.

If a risk of arcing is identified and visualized by the GUI (step 207), the electrodes are grouped such that the critical electrodes are split into separate energy-delivery groups (step 208). Now, in step 209, the GUI displays impedances, AR indexes, IU value and/or CU value of electrodes that are in an acceptable range. If there is no risk of arcing identified step 209 can be directly reached from step 206. Then, in step 210, a PFA treatment is initiated by, e.g. a food pedal of the ablation catheter is continued to be pressed (e.g. by some seconds) by the HCP to the patient if an acceptable positioning of the ablation catheter is shown. Then, in step 211, the procedure continues with step 204 if there was no PFA precheck measurement, with step 212 if the PFA precheck measurement is OK, and with step 213 if the PFA precheck measurement failed. Step 213 contains a repositioning of the catheter, in particular of its ablation portion 12 with respect to the targeted PV antrum. After step 213 the procedure continues with step 202 (see above).

Then, if PFA delivery is aborted by the user in step 212, the procedure continues with step 213 (see explanation of step 213 above). If the PFA delivery is not aborted during treatment, the procedure continues with step 214 the PFA generator provides accurate delivery of ablation energy according to pulse protocol to the user by the electrodes 120 of the ablation portion 12.

According to above procedure, the PFA arcing risk and/or contact uniformity is checked prior PFA ablation in order to guarantee the catheter position with the highest contact uniformity and lowest arcing risk for all electrodes taking part in the PFA. Accordingly, dangerous arcing can be avoided and the electrodes have a uniform contact to the targeted tissue in order to provide high-quality PFA realizing a moat of electrical isolation in one shot.

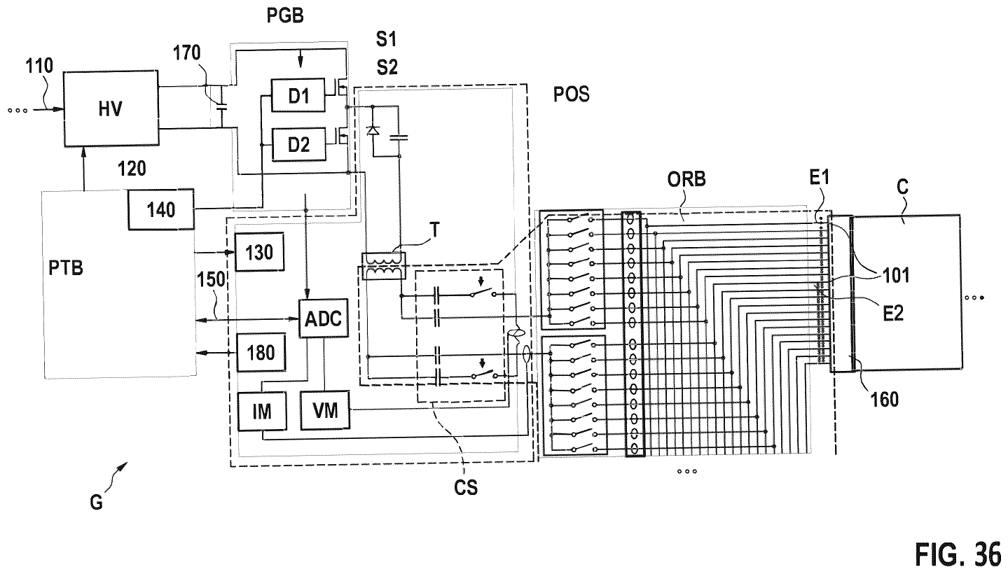

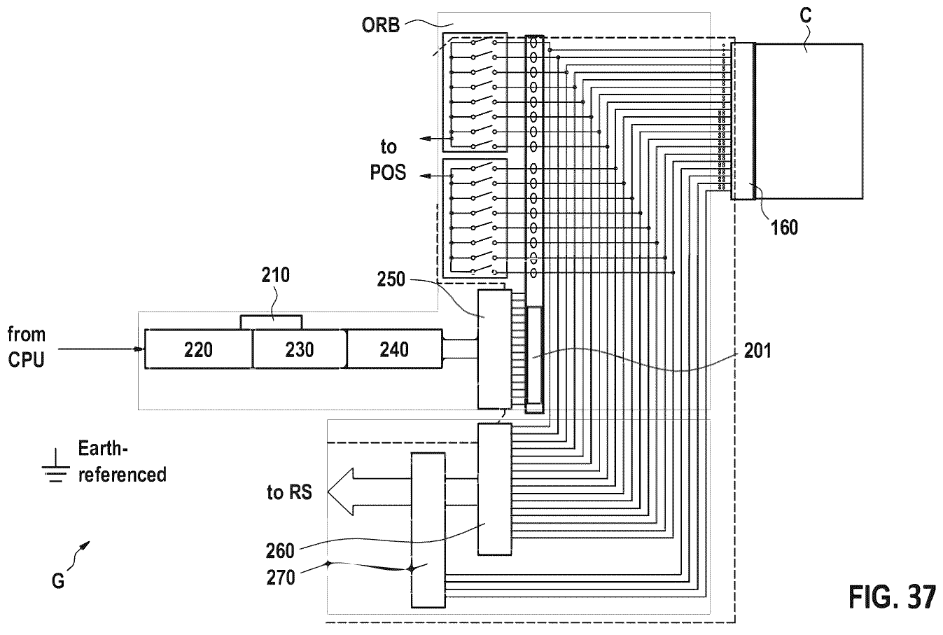

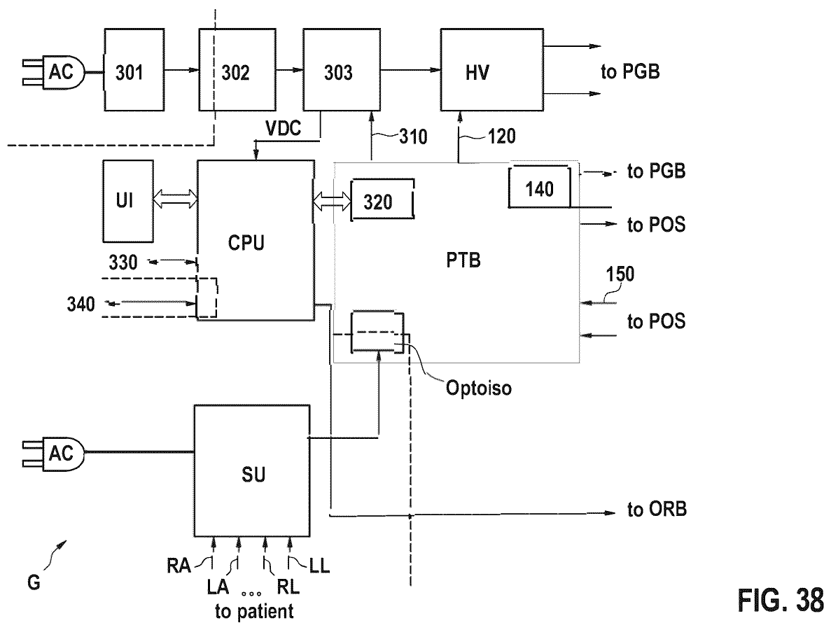

FIG. 36 shows a schematic representation of an exemplary embodiment of a generator G according to the present invention. Notably, generator G of FIG. 36 may also comprise further elements not shown in FIG. 36 (but e.g. shown in FIG. 37 or FIG. 38). As outlined herein, the generator G may be used to generate substantially charge-balanced pulses for a medical device (e.g., for an ablation catheter).

The generator G may comprise a high voltage source HV. The high voltage source HV may provide a voltage of at least 1000 V. For example, the high voltage source may provide a voltage in the range of 1000 V to 4000V. In other examples, the high voltage source may also provide intermediary voltages (e.g., in the range of 100 V to 1000 V) and/or even lower voltages (e.g., in the range of 5 V to 100 V). A supply voltage may be provided via channel 110 to the high voltage source HV which may be increased by the high voltage source HV to provide the desired (high) voltage amplitude.

The generator G may comprise a pulse timing board PTB. The pulse timing board PTB may control the high voltage source HV via channel 120. For example, the pulse timing board PTB may be used to set the voltage amplitude of the high voltage source HV. The pulse timing board PTB may also be used to set the current provided by the high voltage source HV.

The generator may comprise a pulse generation board PGB which may be coupled to the high voltage source HV. The pulse generation board PGB may comprise a high voltage capacitor 170. During operation the high voltage source may charge the high voltage capacitor 170. The high voltage capacitor 170 may thus be used as the energy and/or power source for generating the internal pulse, as described herein.

The pulse generation board PGB may comprise a switching unit. In the example of FIG. 36 the switching unit may comprise a half H-bridge circuit. The half H-bridge circuit of FIG. 36 comprises a first switch S1 and a second switch S2. The switches S1, S2 may be switched via the drivers D1 and D2, respectively. However, also another switching circuit may be used in the generator G (e.g., a H-bridge circuit, or another suitable switching configuration). The drivers D1, D2 of FIG. 36 may be controlled by the pulse timing board PTB via its driver control unit 140 which may be connected to the drivers D1, D2. The switching may enable to generate a reliable internal pulse that may be coupled as an input to the pulse-shaping output stage POS of the generator.

The pulse-shaping output stage (POS) may comprise a transformer T (as described herein) at its input. The transformer T may function as a step-up transformer such that the voltage at the input of the transformer T is increased at the output at the transformer by a defined ratio. Further, the pulse-shaping output stage POS may comprise a capacitor system CS (as described herein). The output of the transformer T may be coupled to the capacitor system CS. The capacitor system CS may comprise various branches with one or more capacitances. The capacitance of a branch may be activated by a switch, as exemplarily indicated in FIG. 36. The switches may be controlled via the capacitor switching unit 130, which may be controlled by the pulse timing board PTB. The generator G may be configured to set at least two total capacitances for the capacitor system CS, as described herein. Notably, the generator may be configured to set a wider plurality of capacitances (e.g., at least three, at least 5, at least 10, at least 50 total capacitances) depending on the chosen circuit of the capacitor system CS. The transformer T and the capacitor system CS may function as the pulse-shaping output stage, as described herein.

The output of the pulse-shaping output stage POS may be coupled to an output relay board ORB of the generator G. The output relay board ORB may couple the output of the pulse-shaping output stage POS in a controlled manner to at least one electrode pair 101 of the medical device C. The medical device C may comprise one or more electrodes (e.g., two electrodes), as outlined herein. The electrode pair may thus be considered a load that can be driven by the generator. In the example, of FIG. 36 each electrode of the medical device C may be individually coupled to the output relay board ORB. As can be seen in FIG. 36, the output relay board ORB is adapted for coupling the paths of sixteen electrodes to the generator, wherein four electrodes are coupled to a different part of the generator (for recording purposes, as described herein). The exemplary electrode pair 101 may comprise the connection of the path of a first electrode E1 (the first of the odd numbered electrodes) with the path of the first of the even numbered electrodes E2. A defined voltage and/or current may thus be applied between the first electrode E1 and the second electrode E2 as the two terminals may be controlled via the generator. Notably, the two terminals are relayed into the pulse-shaping output stage POS such that they may form at least a resistive part of the pulse-shaping output stage.

The output relay board ORB may comprise one or more relay switches to actively couple one or more desired electrode pairs to the pulse-shaping output stage POS. The output relay board ORB may comprise a first relay switch group that may be coupled to a first number of electrodes of the medical device C (e.g., 8 electrodes as indicated in FIG. 36). The first relay switch group may connect the first number of electrodes to a first node of the pulse-shaping output stage depending on the switch configuration. The output relay board ORB may also comprise a second relay switch group that may be coupled to a second number of electrodes of the medical device C (wherein the second number may equal the first number, e.g., 8 electrodes as indicated in FIG. 36). The second relay switch group may connect the second number of electrodes to a second node of the pulse-shaping output stage depending on the switch configuration. Hence, a defined voltage and/or current may be applied between the electrodes associated with the first relay switch group and the electrodes associated with the second relay switch group. Moreover, a substantially charge-balanced pulse may be applied across the electrodes coupled to different relay switch groups.

By coupling the electrode pairs to the pulse-shaping output stage a coupling circuit may be formed, as described herein. The coupled electrode pairs may then function (in total) as a resistive part of the coupling circuit. The coupling circuit may thus also be understood as an RC-circuit, as described herein. The total impedance of the coupled electrode pair may thus be considered as the resistivity R of the RC-circuit. As indicated in FIG. 36 the output relay board may couple various electrode pairs parallel to each other to the pulse-shaping output stage (via the relay switch circuitry). It can also be seen in FIG. 36 that the parallel arrangement of the electrode pairs may be coupled in series to the capacitance of the capacitor system CS. The total impedance of the coupling circuit may thus be defined by a parallel circuit of the electrode pairs. Notably, the capacitor system CS may comprise two capacitor sub systems (as indicated in FIG. 36). The electrode pairs may, for example, be coupled in series between the first capacitor sub system and the second capacitor sub system.

The output relay board ORB may further comprise an interface unit 160 which may facilitate the mechanical and/or communicative coupling of the output relay board ORB to the connector of the medical device C.

Subsequently, the generation of the substantially charge-balanced pulse is discussed. To generate an internal pulse as an input to the pulse-shaping output stage POS the voltage of the high voltage source needs to be applied for a specific time duration. This may be achieved in a controlled way by the switching of the switches S1, S2.

For example, when switch S1 is closed (and switch S2 is open) the high voltage provided by the high voltage capacitor 170 may be coupled to the input of pulse outcoupling board POB of the generator G. For example, the high voltage may then be applied at the transformer T. When switch S1 is open and switch S2 is closed, the voltage at the input of the pulse-shaping output stage POS (e.g., at the input of the transformer T) is actively set to ground. Hence, a defined pulse duration of the internal pulse may be achieved.

As described herein, by coupling the one or more electrode pairs to the pulse-shaping output stage a coupling circuit (e.g., an RC-circuit) is formed. The dynamic response of the coupling circuit to the internal pulse may cause a substantially charge-balanced pulse to be applied in the coupled one or more electrode pairs. Thereby, a substantially charge-balanced pulse is applied via the electrodes, as well. Hence, the coupled medical device C may be used to apply charge-balanced pulses with a high voltage (e.g., onto a tissue for an ablation procedure).

The generator G may further comprise means for measuring the voltage and/or current, e.g., to determine the impedance of the coupled electrode pairs. For example, the pulse-shaping output stage POS may comprise a voltage measurement unit VM. The voltage measurement unit VM may pick up the voltage across the coupled electrode pairs. For example, the voltage may be measured across the parallel circuit of the electrode pair caused by the output relay board ORB (as indicated in FIG. 36). For example, the measured voltage may be associated with the voltage applied across the (total) impedance R of the RC-circuit formed by the coupling circuit.

The pulse-shaping output stage POS may also comprise a current measurement unit IM. The current measurement unit IM may pick up or measure the current in series to the coupled electrodes (e.g., in series to the parallel circuit of the electrode pairs). For example, the measured current may be associated with the current flowing through the RC-circuit (e.g., the current going through the (total) resistivity R).

For enabling an impedance measurement, instead of the described high voltage pulses, e.g. an internal pulse with an intermediary voltage range or a lower voltage range (as described herein) may be applied. This may ensure that the actual high voltage application (which may have a permanent medical effect) is not performed during an impedance measurement. However, during an impedance measurement also a substantially charge-balanced pulse may be triggered in the one or more electrode pairs.

The voltage and/or current measurement data (e.g., of an impedance measurement) may be converted to digital data for an analysis thereof via the analog-to-digital converter ADC of the pulse-shaping output stage POS. Notably, the ADC may also be used to convert analog data of a high voltage sense HV sense of the pulse generation board PG to digital values for further analysis. The measurement results of the voltage measurement unit VM and the current measurement unit IM may be further processed by the generator G. The pulse-shaping output stage POS may also comprise an open circuit shut unit 180. The open circuit shut unit 180 may determine whether an open circuit is present in the coupling circuit (e.g., this may be the case if no electrode pair is actively coupled to the pulse-shaping output stage POS). The presence of an open circuit may be communicated to the pulse timing board PTB which may be configured to not apply an internal pulse if an open circuit is present.

The current and/or voltage measurement results may be used to determine the total impedance of the coupled electrode pairs. To that regard, the voltage drop across all actively coupled electrode pairs may be measured. Furthermore, the current going through the total impedance may be measured by the current measurement unit. Hence, the measurement information may enable to determine an according total impedance. The calculation of the impedance may be performed by the pulse-shaping output stage POS, by the pulse timing board PTB and/or any other suitable processing entity of the generator G (e.g., a processing unit).

For measuring an impedance value of an electrode one electrode may be set as a reference electrode. For example, the second electrode E2 (coupled to the second relay switch group) may function as a reference electrode. Subsequently, each electrode of the first relay switch group may be (separately) activated for a corresponding voltage and current measurement via an applied measurement pulse. For example, a first circuit may be formed between the first and second electrode for an impedance measurement of the first electrode, a second circuit may be formed between the third and fourth electrode for an impedance measurement of the third electrode, and so forth.

The generator G may then analyze the measurement values received from the measurement units VM and IM. Based on the peak voltage and the measured peak current value the impedance value may be determined for each electrode separately. Also, a mean value may be determined for each electrode if the peak current is determined from more than one voltage pulse for each electrode.

The same mechanism may be applied to measure the impedance for the electrodes that are connected to the second relay switch group wherein the reference electrode may be an electrode connected to the first relay switch group, and/or electrode pairs e.g. from the first and second relay switch groups.

Notably, the output relay board ORB may comprise various other switching capabilities. The impedance measurement for the electrodes may also, for example, be performed for a single reference electrode, and separate impedance measurements may be performed for the remaining electrodes.

The information about the impedance of the actively coupled electrode pairs may be used to adjust the total capacitance of the capacitor system CS. For example, a desired RC time constant may be set for the RC-circuit that falls within a desired range. Adapting the time constant may ensure a sufficient control of the evoked charge-balanced pulse (e.g., a desired dynamic response, a desired symmetry of the charge-balanced pulse). As stated, the impedance measurement may be used to determine the total impedance for the actively coupled electrode pairs that may be coupled during a medical treatment. The RC time constant τ may be defined as τ=R·C (wherein R may comprise the total impedance, and C may comprise the total capacitance of the coupling circuit). Accordingly, with a desired RC time constant τ_{S} the (ideal) total capacitance Cₛ to be set may be defined as Cₛ=τ_{S}/R. As it may not always be necessary to have a specific RC time constant value, the total capacitance may be chosen such that the RC time constant falls within a predetermined range. For example, the RC time constant may be chosen to fall in the range of 0.5·10⁻⁶ s to 30·10⁻⁶ s. Notably, the RC time constant may also be chosen to be in the range of 3·10⁻⁶ s to 8·10⁻⁶ s.

The generator G may be configured to automatically set the capacitance of the capacitor system based on the determined total impedance such that the RC time constant may fall within the predetermined range. Alternatively, the generator G may be configured to set a capacitance based on a value of the determined total impedance. For example, if the total impedance may fall in a first range (e.g., between 15 Ohm and 25 Ohm), the generator may set a first total capacitance (e.g., between 0.2 μF and 0.5 μF). If the total impedance may fall in a second range (e.g., between 25 Ohm and 50 Ohm), the generator may set a second total capacitance (e.g., between 0.1 μF and 0.3 μF). If the total impedance may fall in a third range (e.g., between 50 Ohm and 200 Ohm), the generator may set a third total capacitance (e.g., between 0.01 μF and 0.15 μF).

The set total capacitance may then be the total capacitance used in the pulse-shaping output stage when a medical treatment is performed via the electrodes of the medical device C (e.g., an ablation procedure).

FIG. 37 shows a schematic representation of an output relay board ORB of the exemplary embodiment of the generator. FIG. 37 shows the output relay board ORB of FIG. 36 to a fuller extent. The output relay board ORB may comprise a current sensor unit 201. The current sensor unit 201 may comprise one or more current sensors for sensing the current of an electrode. The output relay board ORB may also comprise a channel current measurement unit 240. The channel measurement unit 240 may be communicatively connected to the current sensor unit 201 (e.g., to receive the sensed current of the electrode pairs). The channel current measurement unit 240 may be configured to determine (or measure) the current for each coupled electrode pair based on the sensed current by the current sensor unit 201. The channel current measurement unit 240 may be coupled to a peak detection unit 250. The peak detection unit 250 may be communicatively connected to the current sensor unit 201 (e.g., to receive the sensed current of the electrode pairs). The peak detection unit 250 may be used for determining current peaks (and/or voltage peaks) in the electrode pairs based on the sensed current by the current sensor unit 201. The peak detection unit 250 may be communicatively coupled to the channel current measurement unit 240. For example, the current sensor unit 201, the channel current measurement unit 240 and the peak detection unit 250 may be used to determine the peak current when determining the impedance of an electrode, as described herein.

The output relay board ORB may further comprise an FPGA unit 220. The output relay board ORB may also comprise an ADC unit 230 which may comprise one or more ADCs. The ADC unit 230 may be used to convert the analog signal of the channel current measurement unit 240 to a digital signal for a processing thereof. The FPGA unit 220 and the ADC unit 230 may also be part of a switch relay control unit 210. The switch relay control unit 210 may be configured to control the relays and their configuration within the output relay board ORB (e.g., for closing and/or opening relay switches). For example, the switch relay control unit 210 may control the coupling of the electrode pairs to the pulse-shaping output stage POS. For example, the switch relay control unit 210 may control the relays of the first and second relay switch group.

The output relay board ORB may be controlled by one or more central processing units CPU of the generator G. The one or more central processing units CPU may control the components of the output relay board ORB (e.g., of the switch relay control unit 210). An exemplary central processing unit CPU is shown in FIG. 38.

Coming back to FIG. 37, the output relay board ORB may further comprise a connection to a recording system RS. The output relay board ORB may comprise paths that connect the electrodes (or the electrode channels) of the medical device C to the recording system RS. Hence, the signal of the electrodes may be transmitted to the recording system RS. In another example, the generator G may comprise the recording system RS.

As can be seen in FIG. 37, the output relay board ORB may comprise a first over-voltage protection element 260. The first over-voltage protection element 260 may be for coupling to the electrodes of the medical device C that may perform a high voltage application (e.g., a tissue ablation). The first over-voltage protection element 260 may comprise relay switches that may close or open the path from the signal of the electrodes to the recording system RS. An open relay may not allow any signal to be transmitted from the electrodes (e.g., ablation electrodes) to the recording system RS. Hence, a signal with a high voltage may not be transmitted to the recording system if the relay switches are open during a high-voltage application.

The output relay board ORB may also comprise a second over-voltage protection element 270. The second over-voltage protection element 270 may be also coupled to the first over-voltage protection element 260 to enable additional support for its protective functionality. Notably, the second over-voltage protection element 270 may be directly for coupling to sensing electrodes of the medical device C, as indicated by FIG. 37. The second over-voltage protection element 270 may comprise a transient voltage suppressor.

FIG. 38 shows a schematic representation of various components of the exemplary embodiment of the generator. Notably, the components may be components of the same exemplary generator shown partly in FIG. 36 and FIG. 37. For example, in FIG. 38 the pulse timing board PTB, as well as the high-voltage source HV of FIG. 36 are shown, as well. Notably, the connection to the pulse generation board PGB, the connection to the pulse-shaping output stage POS, the connection to the output relay board ORB of the generator G are shown in FIG. 38.

For generating the voltage for the pulse, the generator G may comprise various stages prior to the high-voltage source HV. For example, the generator G may comprise a power entry module 301 that may be for coupling to an alternating current AC-supply. The power entry module 301 may be coupled to a DC power supply 302. The DC power supply 302 may adapt the alternating current AC supply such that a DC voltage may be provided. For example, the DC power supply 302 may provide a DC voltage of 48 V. The DC power supply 302 may be coupled to a DC-DC converter 303. The DC-DC converter 303 may further increase the DC voltage provided by the DC power supply 302. For example, the DC-DC converter 303 may provide a voltage of 400 V. However, also other voltage may be conceivable (e.g., in the range of 100 V to 800V). The DC-DC converter 303 may be controlled by the pulse timing board PTB (e.g., to set the output voltage of the DC-DC converter 303). The DC-voltage at the output of the DC-DC converter 303 may be provided at the input of the high voltage source HV. The high-voltage source HV may use the provided input voltage to create a high voltage at its output. The output of the high-voltage source may then be coupled to the pulse generation board PGB, as described herein.

The generator G may comprise a central processing unit CPU. The central processing unit may be provided with voltage from the DC-DC converter 303. However, the voltage provided for the central processing unit CPU may be different than the voltage at the output of the DC-DC converter 303. For example, the central processing unit CPU may be provided with 24 V (or 12 V) by the DC-DC converter 303.

The central processing unit CPU may be communicatively connected to various components of the generator G and/or external components. For example, the central processing unit CPU may be communicatively coupled to the pulse timing board PTB and/or to the output relay board OTB. Moreover, the central processing unit CPU may be communicatively coupled to the pulse generation board PGB and to the pulse-shaping output stage POS via the pulse timing board PTB. Hence, the central processing unit CPU may receive various information from components of the generator G and/or may send various instructions to components of the generator G. For example, the central processing unit CPU may receive the current and/or voltage values of an impedance measurement and perform the necessary calculations to determine an according impedance (as described herein).

The central processing unit CPU may be coupled to a user interface UI. The central processing unit CPU may thus receive information from the user interface UI and/or communicate information to the user interface UI for a display thereon. For example, the central processing unit may receive a selection of electrodes of the medical device (which for example, may be inputted by a medical staff) from the user interface UI. The central processing unit CPU may then, for example, communicate instructions to activate the components of the generator G such that the according electrodes are actively coupled (e.g., via the output relay board). The central processing unit CPU may also communicate the results of the impedance measurement (e.g., the total impedance and/or the impedance of the electrodes, as described herein) to the user interface UI. The central processing unit may also perform the necessary calculations to determine what total capacitance should be set in the capacitor system CS by the pulse-shaping output stage. For example, based on determined total impedance the central processing unit CPU may calculate the appropriate total capacitance (or alternatively simply based on the number of actively coupled electrodes). Subsequently, an according control signal may be sent to the pulse-shaping output stage POS to set the appropriate total capacitance. For example, the RC times constants (or the sufficient range of the RC time constants) may also be input via the user interface to the central processing unit. This information may then be used by the central processing unit CPU to enable a setting of the total capacitance with respect to the total impedance and a suitable RC time constant. Additionally or alternatively, these may be stored during manufacture. The user interface may also be used to display the set total capacitance by the generator. Notably, various other technical and/or medical information may be communicated to the user interface for a display thereon.

In an example, the central processing unit CPU may receive a trigger instruction from the user interface UI. For example, the generator G may be for coupling to a synchronization unit SU. The synchronization unit SU may also be comprised by the generator G. The synchronization unit SU may receive an electrocardiogram signal from a patient, preferably, from a patient who receives the medical treatment performed via the medical device C that the generator G supplies the charged balanced pulses to. The synchronization unit SU may receive electrical activities from various electrocardiogram electrode leads, for example from electrodes positioned at the right arm RA, the left arm LA, the right leg RL, the left leg LL. The synchronization unit SU may determine characteristic cardiac events (e.g., an R wave peak, a QRS cycle, etc.). The synchronization unit SU may also provide a trigger signal with the electrocardiogram signal. For example, for an R-wave peak a rising flank may be provided in the trigger signal. The trigger instruction that may be input to the user interface UI may comprise a trigger time interval. After the occurrence of a specific cardiac event the trigger time interval may be the time span after which the internal pulse (or the train of internal pulses) should be generated. This may ensure that the application of the substantially charge-balanced pulses via the electrodes of the medical device C is synchronized to a cardiac event. For example, the time interval may be chosen such that the substantially charge-balanced pulses are applied in a refractory period of the cardiac cycle (and in one or more periods in between two refractory periods, capacitor 170 may be recharged, for example). It may also be conceivable, that the characteristic cardiac event serving as the trigger may be input to the user interface UI or displayed by it. The synchronization unit SU may be directly coupled to the pulse timing board PTB. The electrocardiogram signal and/or its trigger signal may thus be directly communicated to the processing entity that controls the timing of the pulse. The trigger time interval (and/or the according characteristic event) may be stored in the pulse timing board PTB, wherein the trigger time interval was communicated thereto by the central processing unit. Notably, FIG. 38 also indicates the optical isolation of the pulse timing board PTB with respect to the input of the synchronization unit.

In an example, the central processing unit CPU may be configured to determine the characteristic cardiac event within the electrocardiogram signal and may create the trigger signal itself. In that case, the synchronization unit SU may be directly communicatively connected to the central processing unit CPU.

The central processing unit CPU may also comprise various connective capabilities. For example, the central processing unit CPU may comprise a USB connection 330. The central processing unit CPU may also comprise an iso ethernet connection 340.

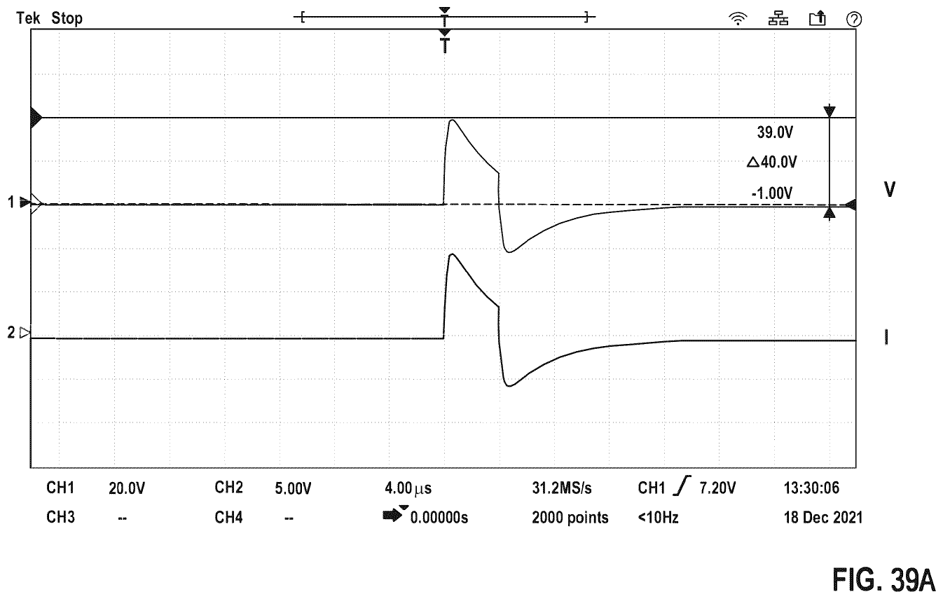

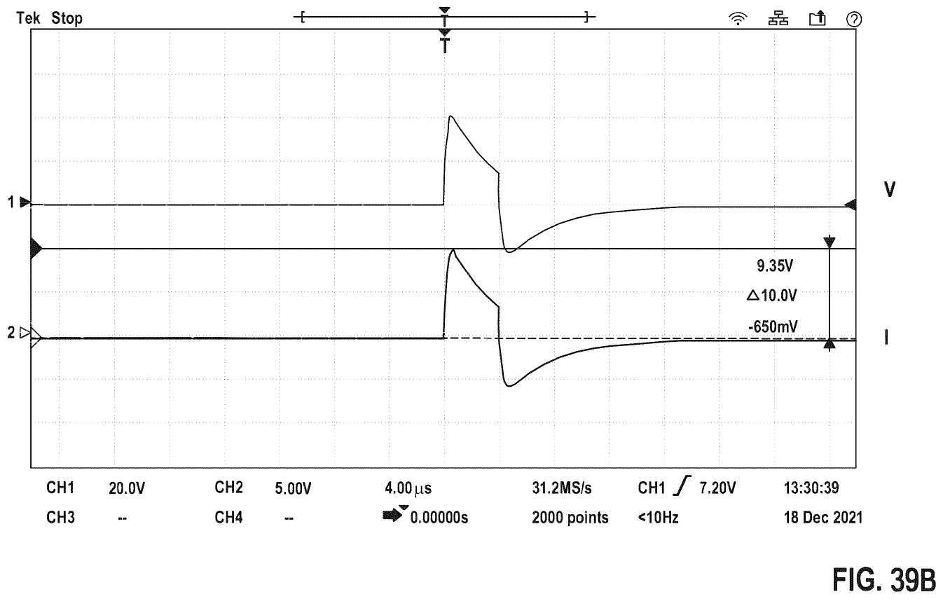

FIG. 39A and FIG. 39B show a representation of a first example of a charge-balanced voltage and current pulse applied by electrodes of the medical device C wherein the pulse is provided to the electrodes by a generator G according to the invention. Channel 1 depicts the pulse voltage V which is scaled down by a factor of 100 (e.g., 40 V on channel 1 represent 4000 V of pulse voltage). Channel 2 depicts the resulting current I displayed as a voltage which is scaled down by a factor of 10 (e.g., 10 V on channel 2 represent 100 A of pulse current). In FIG. 39A and FIG. 39B four electrode pairs were actively coupled to the generator. The total impedance in the coupling circuit was determined as 40 Ohm. The total capacitance was set to 0.13 μF. It can be seen that a biphasic pulse was generated, with a positive and a negative section. Regarding the voltage characteristics, the amplitude of the voltage pulse in this example may peak at about +4000 V and at about −2000V. Regarding the current characteristics, the amplitude of the current pulse in this example may peak at about +100 A and at about −60 A. The exemplary pulse shown on the oscilloscope display may be used as a suitable PFA waveform for a tissue ablation. Due to the circuitry in the generator G a substantially charge-balanced pulse as a PFA waveform can be reliably ensured.

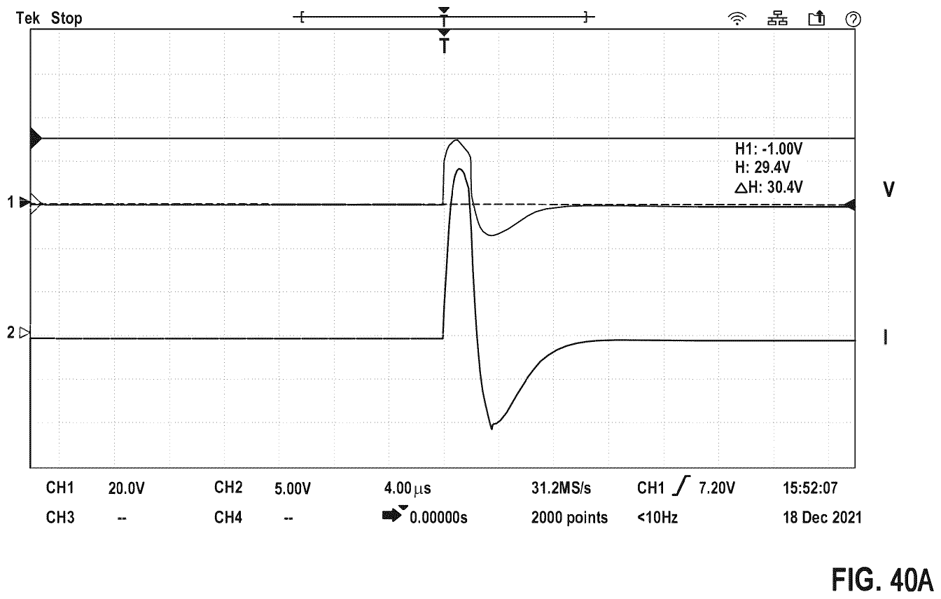

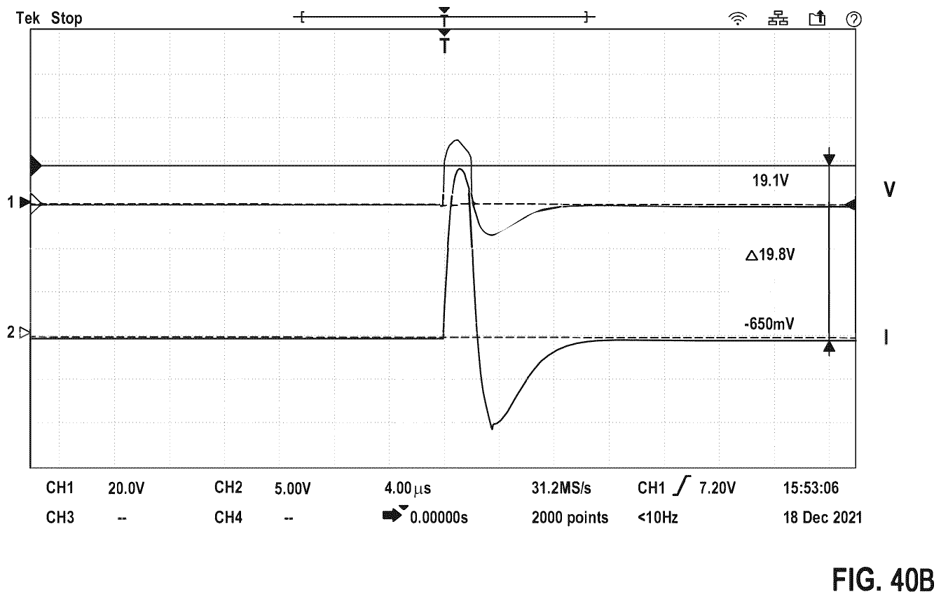

FIG. 40A and FIG. 40B show a representation of a second example of a charge-balanced voltage and current pulse implemented by a generator according to the invention. The channel scaling corresponds to the channel scaling of FIGS. 4a/b. In FIG. 40A and FIG. 40B eight electrode pairs were actively coupled to the generator. In this example, the total impedance was determined as 15 Ohm. The total capacitance was set to 0.34 μF. Regarding the voltage characteristics, the amplitude of the voltage pulse in this example may peak at about +2940 V and at about −1200V. Regarding the current characteristics, the amplitude of the current pulse in this example may peak at about +198 A and at about −100 A. The exemplary pulse shown on the oscilloscope display may also be used as a suitable PFA waveform for a tissue ablation.

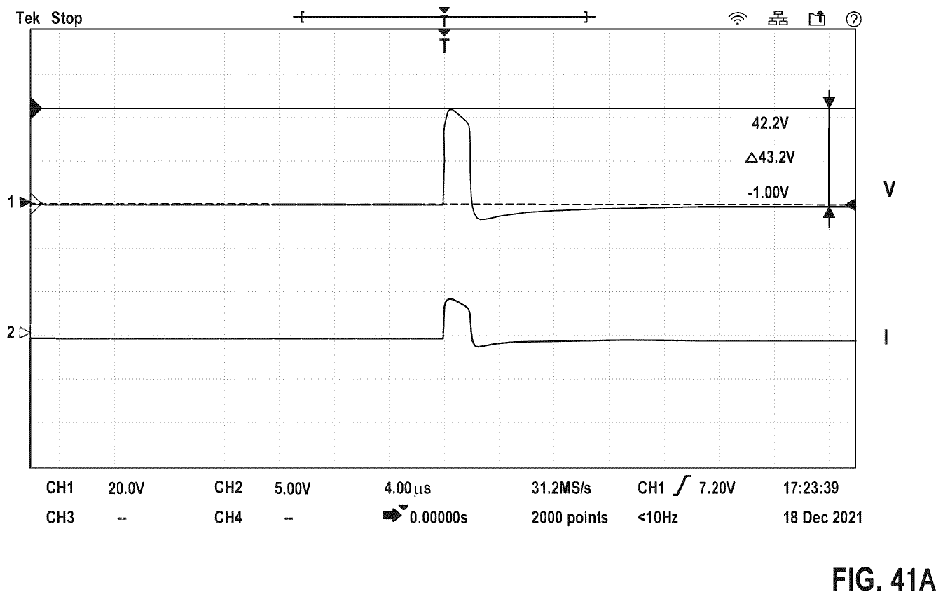

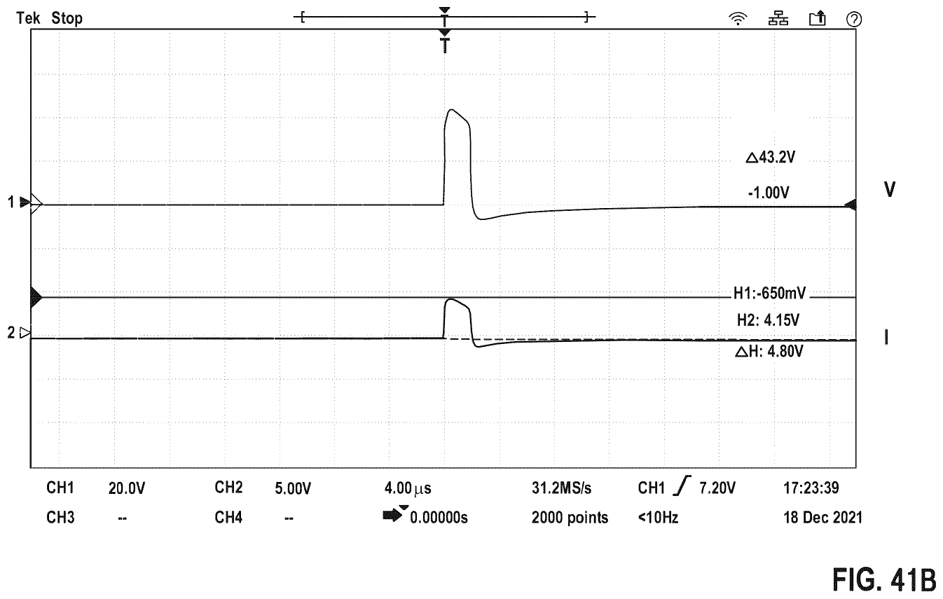

FIG. 41A and FIG. 41B show a representation of a third example of a charge-balanced voltage and current pulse implemented by a generator according to the invention. The channel scaling corresponds to the channel scaling of FIGS. 4a/b. In FIG. 41A and FIG. 41B the total impedance was determined as 90 Ohm. The total capacitance was set to 0.13 μF. Regarding the voltage characteristics, the amplitude of the voltage pulse in this example may peak at about +4320 V and at about −800 V. Regarding the current characteristics, the amplitude of the current pulse in this example may peak at about +41.5 A and at about −15 A. The exemplary pulse shown on the oscilloscope display may also be used as a suitable PFA waveform for a tissue ablation.

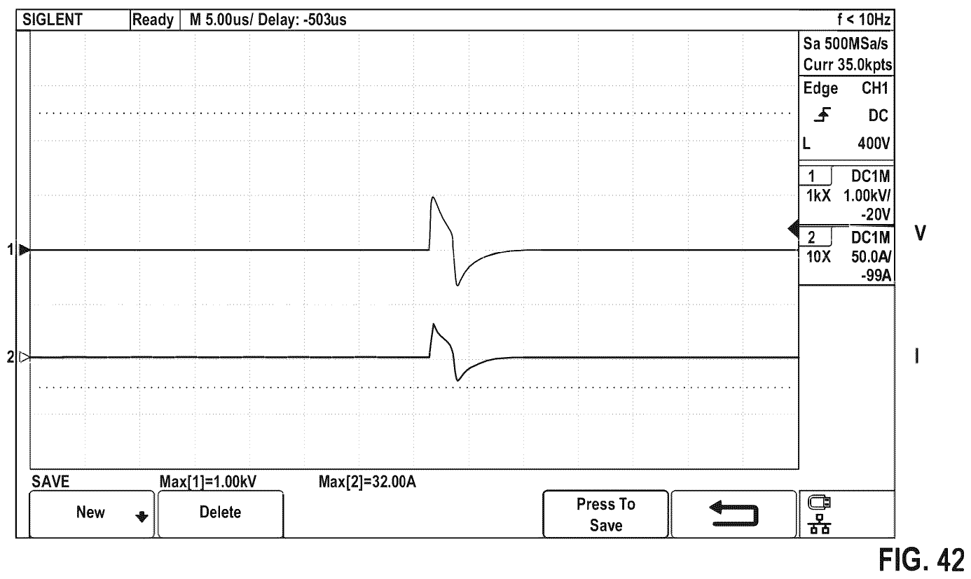

FIG. 42 shows a representation of a fourth example of a charge-balanced voltage and current pulse implemented by a generator according to the invention. In FIG. 42 the total impedance was determined as 90 Ohm. The total capacitance was set to 0.03 μF. This example may illustrate qualitatively the effect of the chosen total capacitance. The total capacitance is comparatively lower to the total capacitance of the example of FIG. 41A and FIG. 41B which, however, comprise the same total impedance. Hence, by adapting the total capacitance the symmetry of the waveform of the substantially charge-balanced pulse may be systematically adjusted.

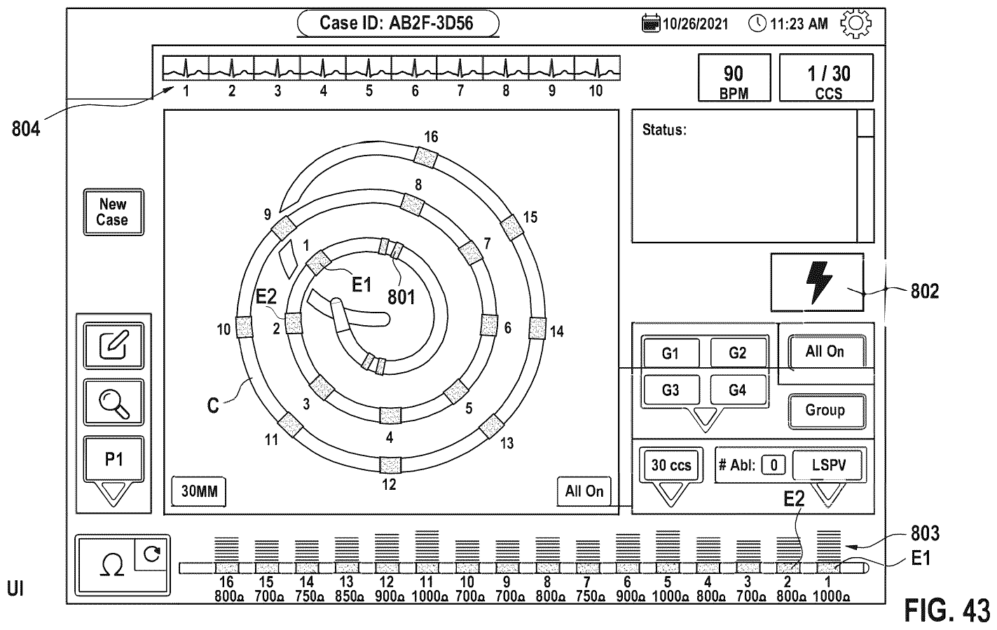

FIG. 43 shows a representation of a user interface UI which may be communicatively coupled to a generator or be part of a generator according to the invention. FIG. 43 may also represent a display of the user interface UI. The user interface UI may be a touchscreen. However, the user interface UI may also be implemented on a monitor wherein an input to the user interface UI may be accomplished via an input device (e.g., a keyboard, a mouse, etc.). The user interface UI may comprise a schematic display of the medical device C. The medical device C in this example may be an ablation catheter. The ablation catheter may comprise various ablation electrodes (e.g., E1, E2), as well as mapping electrodes 801 for sensing and/or mapping purposes (for example, the four lowest electrodes in FIGS. 36 and 37 may correspond to such sensing and/or mapping electrodes). The electrodes of the medical device C may be indicated on the schematic display of the catheter C. The user may select electrodes for active coupling (and thus for a medical treatment) via the user interface. In the example, of FIG. 43 all ablation electrodes were activated for active coupling to the generator. Hence, all electrodes (apart from the purely sensing or mapping electrodes) may be used to facilitate an ablation treatment. The selected electrodes, as well as the number of electrodes may be communicated to the central processing unit CPU. The information may be used, for example, to determine the total impedance (as described herein) and/or the total capacitance to be set in the coupling circuit (as described herein).

At the bottom of the UI, for example, the impedance values for the ablation electrodes of the medical device C may be indicated. For example, the impedance value 803 of the first ablation electrode E1 can be seen in comparison to the other impedance values of the other ablation electrodes. For example, if an electrode impedance is in comparison very high, it may be marked in the user interface UI. High impedances could be attributed to open circuits in the catheter. In such a case the user interface US may not only marks such condition, but the generator may also disallow treatment when a broken electrode is detected this way. In this case the user may have to either disable the broken electrode or exchange the catheter

Also, if an electrode impedance is comparatively low, it may also be accordingly marked in the user interface UI. Low impedances could be attributed to electrodes coming close to another or even touching. This would be a possible arcing and/or bubble formation harzard. In such a case the user interface UI may not only marks such condition, but the generator may also disallow treatment when such a short circuit condition is detected. In this case the user may have to either put the close electrodes into different ablation groups or reposition the catheter. Moreover, there may also be a range of impedances defined where treatment is allowed but a warning is presented on the user interface UI that electrodes are close.

The critical impedance threshold (or a critical deviation) may be communicated to the user interface from the generator (e.g., from its central processing unit CPU). Published WO 2022/159665 of the applicant discloses several ways to determine critical impedances. The content of this application is hereby fully incorporated. The user interface may comprise an activation button 802 that may initiate the procedure for applying the substantially charge-balanced pulses via the electrodes. The user interface UI may also indicate an electrocardiogram signal 804 of the patient who may receive the treatment by the medical device C. The user interface UI may also comprise a trigger panel. Upon activation of the trigger panel the trigger time interval with respect to a characteristic cardiac event (e.g., an R wave) may be set. The user interface UI may also comprise an impedance measurement panel. Upon activation of the impedance measurement panel, an impedance measurement may be triggered (e.g., a measurement of the total impedance and/or a measurement of the electrode's impedances). The user interface UI may also comprise a capacitor panel. Upon activation of the capacitor panel, a total capacitance of the capacitor system may be manually set by the user. The activation of the capacitor panel may also trigger the central processing unit CPU to determine an according total capacitance based on the selected electrodes. To that regard, the activation of the capacitor panel may also trigger a measurement of the total impedance. The user interface UI may also comprise an RC time constant panel. Upon activation of the RC time constant panel an RC time constant and/or a range of suitable RC time constants for the coupling circuit may be inputted by the user.

It is noted that the above examples may be combined with further aspects as described herein and details of the examples may also be omitted, as will be understood by the skilled person. For example, numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

What is claimed is:

1. An ablation system for treatment of patient tissue, comprising:
an ablation catheter comprising an ablation portion having a plurality of electrodes at a distal end, wherein the ablation portion comprises at least two loop sections forming a three dimensional spiral,
a generator adapted to connect to a proximal end of the ablation catheter to electrically couple to the plurality of electrodes, the generator configured to energize one or more of the plurality of electrodes with high-voltage charge-balanced pulsed electric fields and receive electrical signals from one or more of the plurality of electrodes; and
the generator comprising an interface configured to connect to an external device and further configured to protect the external device from a high voltage pulse.

2. The ablation system of claim 1, wherein the external device comprises a mapping system.

3. The ablation system of claim 1, wherein the external device comprises a recording system.

4. The ablation system of claim 1, wherein the external device comprises an electro-anatomical mapping system.

5. The ablation system of claim 4, wherein the electro-anatomical mapping system comprises a recording system.

6. The ablation system of claim 1, wherein the interface is configured to protect the external system from a high voltage pulse.

7. The ablation system of claim 6, wherein the interface comprises one or more voltage suppressors configured to protect the external system from a high voltage pulse.

8. The ablation system of claim 6, wherein the interface comprises one or more switches configured to protect the external system from a high voltage pulse.

9. The ablation system of claim 6, wherein the interface comprises one or more relays configured to protect the external system from a high voltage pulse.

10. The ablation system of claim 1, wherein the generator further comprises an electronic control unit adapted to switch between an ablation mode and a mapping mode for each of the plurality of electrodes.

11. The ablation system of claim 10, wherein electrodes used for ablation in the ablation mode are used for mapping in the mapping mode.

12. The ablation system of claim 1, wherein a distance between a first electrode on a first loop section having a first polarity and a second electrode on a second loop section having a second polarity opposite the first polarity and closest to the first electrode results in an arcing risk index less than 0.25 in a compressed position where the ablation portion is flattened by an external force and an arcing risk index less than 0.25 in an uncompressed position where the ablation portion is not restricted by any external force.

13. The ablation system of claim 1, wherein a distance between a first electrode on a first loop section having a first polarity and a second electrode on a second loop section having a second polarity opposite the first polarity and closest to the first electrode results in an arcing risk index less than 0.15 in a compressed position where the ablation portion is flattened by an external force and an arcing risk index less than 0.15 in an uncompressed position where the ablation portion is not restricted by any external force.

14. An ablation system for treatment of patient tissue, comprising:
an ablation catheter comprising an ablation portion having a plurality of electrodes at a distal end, wherein the ablation portion comprises at least two loop sections forming a three dimensional spiral,
a generator adapted to connect to a proximal end of the ablation catheter to electrically couple to the plurality of electrodes, the generator configured to energize one or more of the plurality of electrodes with high-voltage charge-balanced pulsed electric fields and receive electrical signals from one or more of the plurality of electrodes; and
an external device, wherein the generator comprises an interface configured to connect to the external device further configured to protect the external device from a high voltage pulse.

15. The ablation system of claim 14, wherein the external device comprises a mapping system.

16. The ablation system of claim 14, wherein the external device comprises a recording system.

17. The ablation system of claim 14, wherein the external device comprises an electro-anatomical mapping system.

18. The ablation system of claim 17, wherein the electro-anatomical mapping system comprises a recording system.

19. The ablation system of claim 14, wherein the interface is configured to protect the external system from a high voltage pulse.

20. The ablation system of claim 19, wherein the interface comprises one or more voltage suppressors configured to protect the external system from a high voltage pulse.

21. The ablation system of claim 19, wherein the interface comprises one or more switches configured to protect the external system from a high voltage pulse.

22. The ablation system of claim 19, wherein the interface comprises one or more relays configured to protect the external system from a high voltage pulse.

23. The ablation system of claim 14, wherein the generator further comprises an electronic control unit adapted to switch between an ablation mode and a mapping mode for each of the plurality of electrodes.

24. The ablation system of claim 10, wherein electrodes used for ablation in the ablation mode are used for mapping in the mapping mode.

25. The ablation system of claim 14, wherein a distance between a first electrode on a first loop section having a first polarity and a second electrode on a second loop section having a second polarity opposite the first polarity and closest to the first electrode results in an arcing risk index less than 0.25 in a compressed position where the ablation portion is flattened by an external force and an arcing risk index less than 0.25 in an uncompressed position where the ablation portion is not restricted by any external force.

26. The ablation system of claim 14, wherein a distance between a first electrode on a first loop section having a first polarity and a second electrode on a second loop section having a second polarity opposite the first polarity and closest to the first electrode results in an arcing risk index less than 0.25 in a compressed position where the ablation portion is flattened by an external force and an arcing risk index less than 0.25 in an uncompressed position where the ablation portion is not restricted by any external force.
